(19) United States
(12) Patent Application Publication
(10) Pub. No.: US 2011/0022129 A1
(43) Pub. Date: Jan. 27, 2011

(54) NANOPARTICLES FOR PHOTODYNAMIC THERAPY

(76) Inventors: Robert K. Prud'homme, Lawrenceville, NJ (US); Marian Gindy, Haledon, NJ (US); Baris Ungun, San Carlos, CA (US); Stephanie Budijono, Jakarta (ID); Ying Liu, Chicago, IL (US)

Correspondence Address: MEDLEN & CARROLL, LLP, 101 HOWARD STREET, SUITE 350, SAN FRANCISCO, CA, 94105, US

(21) Appl. No.: 12/741,334

(22) PCT Filed: Nov. 5, 2008
(86) PCT No.: PCT/US08/12485

§ 371 Date: Sep. 30, 2010

Related U.S. Application Data
(60) Provisional application No. 61/001,839, filed on Nov. 5, 2007.

(51) Int. Cl. A61N 5/06; C12N 13/00

(52) U.S. Cl. 607/88; 435/173.1; 977/773; 977/904

(57) ABSTRACT

Embodiments of the invention include nanoparticulate products of a flash nanoprecipitation process, the products comprising, in combination, pre-formed nanoparticles having properties of an upconversion phosphor and a hydrophobic organic compound having the properties of a photosensitizer, and a method of photodynamic therapy comprising the use of the nanoparticulate products is described.

### FIELD

Embodiments of the invention described herein relate to the field of photodynamic therapy. In particular, some embodiments relate to photodynamic therapy administered by means of nanoparticles comprising preformed, size-controlled colloidal particles having the properties of an upconversion phosphor, co-encapsulated with one or more molecularly dissolved hydrophobic compounds having the properties of a photosensitizer.

### BACKGROUND

Photodynamic therapy refers to the treatment of tumors by administration of a photosensitizing compound or its metabolic precursor. The photosensitizing compound is delivered by one means or another to the proximity of a tumor, creating a photosensitized target. When the photosensitizing compound is exposed to light of appropriate wavelength, a process that damages or kills nearby tumor cells ensues. Since the light needed to excite most photosensitizers cannot penetrate far into biological tissue, photodynamic therapy has been limited to treatment of tumors growing on or just under the skin or on mucosal epithelia where light can be optically pumped to the treatment site. Not only is delivering light to the photosensitizer a limitation, delivering photosensitizers to tumors is complicated by the hydrophobicity of most photosensitizers (generally porphyrins, chlorines, and phthalocyanines). Besides making delivery via aqueous extracellular fluids difficult, hydrophobic photosensitizers work better in hydrophobic than in aqueous environments. Additionally, delivery of a photosensitizer to a tumor implies delivery to the whole body, including the body's surface, which remains sensitive to light until the agent is cleared from the patient's system.

Current treatment options for many cancers such as chemotherapy and radiation therapy have shown less than promising outcomes. This is especially true of lung cancer. The standard treatment for patients with stage IIIB non-small-cell lung carcinoma (“NSCLC”) is non-operative (Komaki et al., Inst. J. Radiat. Oncol. Biol. Phys. 2000, 48: 113; Furose et al., Journal of Clinical Oncology 1999, 17:2692-2699; Reyes et al., Journal of Thoracic and Cardiovascular Surgery 1991, 101:946-947). Patients with a good performance status are treated with chemotherapy but are rarely rendered disease-free. The survival of NSCLC patients with pleural spread is reported to be in the range of 6 to 9 months for patients treated with the non-operative standard of care compared to a median survival of 21.7 months for patients in a stage II PDT clinical trial for NSCLC (Friedberg et al., Journal of Clinical Oncology 2004, 22:2192-2201; Komaki et al., 2000; Furose et al., 1999).

Photodynamic therapy is an evolving new treatment for cancer, among many other indications, but the true potential of this therapy has been hindered by the limitations mentioned above.

Therefore, current research in the field has three areas of focus: (i) enabling therapy with deeper-penetrating longer wavelength light, (ii) targeting the therapeutic molecules to ensure that they will have greater affinity for tumor cells than healthy cells, and (iii) finding effective hydrophilic photosensitizers or finding ways to solubilize hydrophobic photosensitizers in aqueous media.

### SUMMARY

In one aspect, the invention is embodied in a method comprising:

a) providing
(i) a composite nanoparticle comprising an upconversion phosphor and a photosensitizer,
(ii) a source of infra-red light, and
(iii) a cell

b) contacting the cell with the nanoparticle to create a contacted cell, and

c) exposing the contacted cell to infra-red light from the light-source.

In one embodiment, the nanoparticle used in the method further comprises a ligand, which ligand binds to the cell in step b).

In one embodiment, the nanoparticle is capable of entering the nanoparticle enters said cell.

In one embodiment, after step b), the composite nanoparticle releases an active oxygen species.

In one embodiment, the cell is in a live animal, which may be a human.

In a preferred embodiment, the cell is a cancer cell.

In one embodiment, the animal or human is suspected of having non-small cell lung cancer.

In one aspect, the invention is embodied in a system for killing cancer cells in vivo comprising:
a) an infrared-activable photosensitizer incorporated in a composite nanoparticle, the nanoparticle having an outer surface to which is attached a ligand,
b) an upconversion phosphor incorporated in the nanoparticle,
c) a means for contacting the nanoparticle to a cancer cell, the cell having a binding site for the ligand, and
d) a means for exposing the cell to an infra-red light.

In one embodiment, the ligand is specific for a neuroendocrine receptor.

In one embodiment, the ligand is luteinizing hormone releasing hormone (“LHRH”).

In one aspect, the invention is embodied in a composite nanoparticle comprising a hydrophobic organic molecule, an upconversion phosphor, a photosensitizer and an amphiphilic polymer.

### BRIEF DESCRIPTION OF THE FIGURES

FIG. 1 is a schematic of a process for preparing multicomponent (“composite”) nanoparticles by flash nanoprecipitation in a multi-inlet vortex mixer, and a product thereof.

FIG. 2 shows the relationship between the size (hydrodynamic diameter) of PEG-b-PCL-encapsulated (“protected”) gold (“Au”) nanoparticles and the amount of gold loaded therein. Figure inset demonstrates that size depends on the cubic root of Au volume fraction (φ_{Au}).

FIG. 3 compares size distributions of PEG-b-PCL protected Au nanoparticles at various Au loadings (in weight percent in final solution), and relative to polystyrene latex spheres.

FIG. 4 is a transmission electron micrograph of PEG-b-PCL (5,000-b-6,000 g/mol)-protected Au nanoparticles prepared at a loading of 23.3 wt % Au.

FIG. 5A is a photograph of aqueous dispersions of PEG-b-PCL (5,000-b-6,000 g/mol) nanoparticles encapsulating Au (9.4 wt %) and β-carotene (30.2 wt %), prepared via flash nanoprecipitation (vials 1 and 2) compared with an ordinary (unprotected) gold colloid (vial 3). Vial (1) contains an unfiltered dispersion, vials (2) and (3) contain filtered dispersions.

FIG. 5B compares the light absorption spectra of the dispersions in vials (1) and (2).

FIG. 6 compares particle size distributions of freshly made PEG-b-PCL (5,000-b-6,000 g/mol) nanoparticles encapsulating Au (9.4 wt %) and β-carotene (30.2 wt %) in 155 mM NaCl (solid line) and nanoparticles stored 28 days (dashed line) at room temperature.

FIG. 7 shows that increasing amounts of the encapsulating polymer in a PEG-b-PCL-protected Au nanoparticle do not add to the size of the particle until the polymer component accounts for more than about 33% of the particle's volume.

FIG. 8 shows, for various concentrations of gold in solution, the size distribution of gold aggregates in the flash nanoprecipitation product (as formed in about 40 milliseconds), where the “size” of an aggregate is determined by the number of gold monomers in the aggregate.

FIG. 9 shows UV-visible absorbance spectra of dodecane capped Au (C₁₂—Au) nanoparticles dispersed in THF (solid line) and PEG-b-PCL protected C₁₂—Au nanoparticles dispersed in 1:10 v/v THF:water (dashed line).

FIG. 10 is a graphical representation of the solubility of β-carotene and a block copolymer stabilizer as a function of THF concentration at 35° C.

FIG. 11 is a photomicrograph a composite nanoparticulate product.

FIG. 12 shows energy level diagrams of the Er3+, Tm3+ and Yb3+ dopant ions and up-conversion mechanisms following 980 nm diode excitation. The full, dotted, and curly arrows represent emission, energy transfer, and multi-phonon relaxation processes, respectively.

FIG. 13(a) is a TEM of SiO2 coated nanoparticles, approximately 100 nm.

FIG. 13(b) indicates the luminescence of the suspension at 100 mW.

FIG. 13(c) is a graphical representation of up-conversion spectra of the nanoparticles.

FIG. 14 is a photograph showing externally activated photodynamic therapy delivered to a mouse bearing a flank tumor of malignant mesothelioma.

FIG. 15 Compares tumor growth in control mice and in mice treated with infrared light and photosensitizer-containing nanoparticles, “empty” nanoparticles with and without infrared light, photosensitizer delivered without nanoparticles (with and without infra-red light), and infra-red light only.

FIG. 16 Shows in vivo survival analysis for nanophosphor-mediated photodynamic therapy via the regimes of FIG. 15.

FIG. 17 shows the size distribution of composite nanoparticles stabilized by PEG-PCL.

FIG. 18 shows SEM images of PEG-PCL protected CNPs at (a) low magnification and (b) high magnification

FIG. 19 shows optical and infra red images of PEGylated CNPs incubated in AB12 mesothelioma cells.

FIG. 20 shows bleaching of ADPA, which signifies production of singlet oxygen by IR-illuminated CNPs.

FIG. 21 shows size distribution of PEG-PLGA protected CNPs.

FIG. 22 shows SEM image of nanometer-sized PEG-PLGA protected CNPs.

FIG. 23 shows UV-Vis absorbance of mTPP in different phases post-centrifugation.

FIG. 24 shows TGA data on the amount of UCNPs in the pellet (CNPs) phase.

### DETAILED DESCRIPTION

Embodiments of the present invention overcome photodynamic therapy's limitation to surface applications by allowing infra-red light to indirectly excite or activate photosensitizers, most of which are excited only by visible light that cannot reach tumors unless they are growing on or just under the skin. In preferred embodiments, an upconversion phosphor absorbs the long-wavelength light and converts it into shorter wavelength visible light. Table 2 is a non-limiting list of exemplary upconversion phosphors. Some embodiments also circumvent the problem of delivering hydrophobic photosensitizers (porphyrins, chlorines, and phthalocyanines, the most apt photosensitizers, are all hydrophobic) via aqueous extracellular fluids. Table 3 provides a non-limiting list of exemplary photosensitizers. Although the problem can be remedied by adding side chains to the photosensitizer or enrobing it in a liposome, for example, it is preferable to allow the photosensitizer to remain in a hydrophobic environment, where it will work better than in an aqueous environment. Moreover, “hydrophilized” photosensitizers must be administered at doses high enough to account for dilution in the entire volume of the body's aqueous phase. Embodiments of the invention also provide co-delivery and co-localization of the phosphor and the photosensitizer, circumventing the inefficiency of injecting the two agents separately. Further, some embodiments are specifically targeted to tumor cells or to the tumor's vasculature. Thus, the photosensitizer does not recirculate unabated throughout the whole body. Accordingly, the body's surface does not remain as sensitized to light for as long as it otherwise would, an important consideration inasmuch as the sensitizer generates highly toxic species of oxygen.

The various embodiments of the invention provide a means for delivering photosensitizers to the proximity of tumors in combination with a material that absorbs long-wavelength light and converts it to visible light. The two components are carried together in a composite nanoparticle of the kind described in detail infra. The composite nanoparticles are manufactured by a process comprising the steps of providing a hydrophobic organic compound dissolved in a solvent, a hydrophobic inorganic nanoparticle dispersed in the same or a compatible solvent, and an amphiphilic polymer dissolved in the same or a compatible solvent, and micromixing the above with an anti-solvent such that composite nanoparticles form. The inorganic nanoparticle is rendered hydrophobic by means of a surface modification made either during the synthesis of the particle or be treating the surface of the synthesized particle. The amphiphilic copolymer stabilizes the organic and inorganic components and effectively encapsulates those components in a hydrophilic shell. The composite product is itself a nanoparticle but is hydrophilic so it disperses stably in aqueous media.

The amphiphilic polymer may comprise diblock copolymers having a hydrophobic block and a hydrophilic block selected to permit controlled stabilization of the encapsulated agents. Or, the amphiphilic polymer may have a multiplicity of blocks along the backbone, or hydrophilic grafted chains attached at multiple sites along a hydrophobic polymer backbone, or the converse with a hydrophilic backbone and hydrophobic grafted chains.

Typically, the stabilizing amphiphilic polymer is a copolymer of a hydrophilic block coupled with a hydrophobic block. Nanoparticles formed by the process of this invention can be formed with graft, block or random amphiphilic copolymers. These copolymers can have a molecular weight between 1000 g/mole and 50,000 g/mole, or preferably between about 3000 g/mole to about 25,000 g/mole, and more preferably at least 2000 g/mole. Alternatively, the amphiphilic copolymers used in this invention exhibit a water surface tension of at least 50 dynes/cm² at a concentration of 0.1 wt %.

Examples of suitable hydrophobic blocks in an amphiphilic copolymer include but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinyllimidazole; aminoalkyls including aminoalkylacrylates, aminoalkylsmethacrylates, and aminoalkyl(meth)acrylamides; styrenes; cellulose acetate phthalate, cellulose acetate succinate, hydroxypropylmethylcellulose phthalate, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery Wright, Bristol, 1987; Arshady, J. Controlled Release 17:1-22, 1991; Pitt, Int. J. Phar. 59:173-196, 1990; Holland et al., J. Controlled Release 4:155-0180, 1986); hydrophobic peptide-based polymers and copolymers based on poly(L-amino acids) (Lavasanifar, A., it al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anyhydride copolymers of vinyl methylether and other vinyl ethers, polyamides (nylon 6,6), polyurethane, poly(ester urethanes), poly(ether urethanes), poly(ester-urea), Particularly preferred polymeric blocks include poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly (caprolactone) or poly(lactic acid) For non-biologically related applications particularly preferred polymeric blocks include polystyrene, polyacrylates, and butadienes. Natural products with sufficient hydrophobicity to act as the hydrophobic portion of the amphiphilic polymer include: hydrophobic vitamins (for example vitamin E, vitamin K, and A), carotenoids and retinols (for example beta carotene, astaxanthin, trans and cis retinal, retinoic acid, folic acid, dihydrofolate, retinylacetate, retinyl palmintate), cholecalciferol, calcitriol, hydroxycholecalciferol, ergocalciferol, alpha-tocopherol, alpha-tocopherol acetate, alpha-tocopherol nicotinate, and estradiol. The preferred natural product is vitamin E which can be readily obtained as a vitamin E succinate, which facilitates functionalization to amines and hyroxyls on the active species.

Examples of suitable hydrophilic blocks in an amphiphilic copolymer include but are not limited to the following: carboxylic acids including acrylic acid, methacrylic acid, itaconic acid, and maleic acid; polyoxyethylenes or poly ethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-crrylamideo-2-methylpropane sulfonic acid and styrene sulfonate, polyvincyl pyrrolidone, starches and starch derivatives, dextran and dextran derivatives; polypeptides, such as polylysines, polyarginines, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids. The particularly preferred hydrophilic blocks are poly ethylene oxide and poly poly hydroxyl propyl acrylamide and methacrylamide to prepare neutral blocks since these materials are in currently approved medical applications. To prepare anionic copolymers acrylic acid and methacrylic acid and poly aspartic acid polymers are especially preferred. And to produce cationic amphiphilic copolymers DMAEMA (dimethylaminoethylmethacrylate), polyvinyl pyridine (PVP) or dimethylaminoethylacrylamide (DMAMAM).

Preferably the blocks are either diblock or triblock repeats. Preferably, block copolymers for this invention include blocks of polystyrene, polyethylene, polybutyl acrylate, polybutyl methacrylate, polylactic acid (PLA), polyglutamic acid (PGA) and PLGA copolymers, polycaprolactone, polyacrylic acid, polyoxyethylene and polyacrylamide. A listing of suitable hydrophilic polymers can be found in Handbook of Water-Soluble Gums and Resins, R. Davidson, McGraw-Hill (1980).

In graft copolymers, the length of a grafted moiety can vary. Preferably, the grafted segments are alkyl chains of 4 to 18 carbons or equivalent to 2 to 9 ethylene units in length. In addition, the grafting of the polymer backbone can be useful to enhance solvation or nanoparticle stabilization properties. A grafted butyl group on the hydrophobic backbone of a diblock copolymer of a polyethylene and polyethylene glycol should increases the solubility of the polyethylene block. Suitable chemical moieties grafted to the block unit of the copolymer comprise alkyl chains containing species such as amides, imides, phenyl, carboxy, aldehyde or alcohol groups.

Some embodiments of the present invention incorporate upconversion phosphors and, in some embodiments, a photosensitizer is incorporated in the same particle with the phosphor. Some embodiments additionally incorporate one or more imaging agents, drugs, and/or “tags” that emit detectable signals for monitoring and tracking the particles. Optionally, the phosphor, the photosensitizer and the surface of the shell may, independently, be modified with a targeting molecule. Liposomes can be similarly modified but do not capture upconversion phosphors effectively, so the advantage of exciting with infra-red light is lost. Silica shells accommodate upconversion materials and photosensitizers, but the photosensitizers are not highly loadable into silica shells.

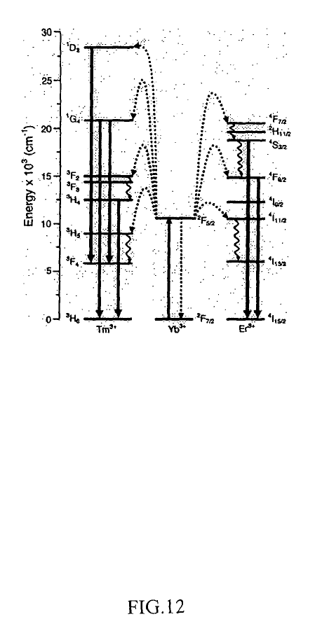

In some embodiments, the present invention provides a system comprising an upconverting (or “upconversion”) phosphor and a photosensitizer in operable combination such that the photosensitizer can be stimulated with long-wavelength light. Up-converting phosphors are ceramic materials in which rare earth atoms are embedded in a crystalline matrix. The crystals can range from a few nanometers (“nanophosphors”) to tens of microns. Nanophosphors are of interest in embodiments of the present invention, in part because they are programmable (i.e., under the control of the artisan) with respect to wavelength, emission spectrum, lifetime, size, and efficiency (Murray et al., Journal of the American Chemical Society 1993, 115:8706-8715; Bhargava, Journal of Luminescence 1996, 70: 85-94; Tapec et al., Journal of Nanoscience and Nanotechnology 2002, 75:282-288). Because of the ‘tunable’ nature of nanophosphors, a wavelength especially suited to penetrating a given medium (such as human tissue) can be chosen to illuminate the phosphor, and the phosphor, in turn, can be chosen to emit, at that excitation, an optimal treatment or diagnostic wavelength. These rare-earth nanophosphors are non-toxic. While embodiments of the invention can be practiced without knowledge of any mechanism, up-conversion can be described as either sequential excitation of the same atom or simultaneous excitation of two centers, followed in either case by relaxation with an energy transfer (Li et al., Journal of Physical Chemistry B, 2006, 110:1121-1127; Qin et al., Applied Physics Letters 2007, 90:1-3). These up-conversion phenomena are diagrammed in FIG. 12. The emissions of upconversion nanophosphors consist of sharp lines characteristic of atomic transitions in a well-ordered matrix. By using different rare earth dopants, a large number of distinctive emission spectra can be obtained (Auzel, Chemical Reviews, 2004, 104:139-173; Nelson et al., Chemistry of Materials 2003, 15:688-693). The emission spectra of the nanophosphors can be tailored to match the absorption/excitation wavelengths of many photosensitizers activated by visible light.

These features, combined with their long fluorescence lifetimes—up to several ms—make upconverting materials very desirable in many applications including, in principle, photodynamic therapy. There is at least one instance of an upconverting organic dye being paired with a photosensitizing compound (Friedberg et al., Annals of Thoracic Surgery 2003, 75:952-959), but coupling upconverting rare-earth species and photosensitizers as a means of delivering light to the sensitizer has, to Applicants' knowledge, been unsuccessful, probably due at least in part to the poor aqueous dispersibility of such upconverters.

Although a handful of organic dyes display an upconverting capability, most upconverting materials are lanthanides, uranides, or transition metal ions. Most frequently used are the trivalent ionic lanthanide species, hosted in a semiconductor or bulk oxide or fluoride lattice, or chelated with an organic compound. Typically, the doped solids display the greatest number of emission events for a given amount of impinging light (“quantum yield”), and have superior brightness. The nanocrystalline form of such doped solids shows significant advantages in efficiency and brightness over the microcrystalline form. Thus, it is of interest to be able to produce and use nanocrystalline rare-earth ion-doped solids as upconversion phosphors.

Frequently mentioned target applications for upconversion phosphors include: inks (particularly for security applications); particles for displays; particles sensitive to infrared radiation, heat, or chemicals that are relevant in biological or non-biological sensors, and particles for biological labeling. For almost all of these applications, whether for ink, or to spray, coat, or otherwise apply to other surfaces, some kind of stable dispersion of the particles is required Otherwise, the particles will aggregate, effectively restricting their use to solid or larger forms of the material. In addition, for many applications, the nanoparticles should be robust and pH-tolerant in aqueous media. To Applicants' knowledge, no available method makes products that meet all of these criteria, except for the method of flash nanoprecipitation as described (with modifications disclosed herein) in Johnson et al., Australian Journal of Chemistry 2003, 56: 1021-1024, and in U.S. Patent Application Publication No. 2004/0091546 and International Publication No.: WO 2006/014626.

A nanoparticle for photodynamic therapy should be water-soluble but should have a highly loadable hydrophobic core if it is to deliver the most effective photosensitizers, which are hydrophobic; the particle's outer surface should be easily modified with antibodies or other ligands to ensure tissue-specific instead of systemic delivery; a material should be present in the nanoparticle to capture therapeutic irradiation at a deeply tissue-penetrating wavelength (a wavelength of around 1 micron will readily penetrate to a depth of a centimeter) and to upconvert the radiation to the visible, solely within the local microscopic environment of the photosensitizer; finally, the nanoparticle should incorporate a (smaller) nanoparticle, preferably a radio-opaque or paramagnetic (preferably superparamagnetic) metal, to allow the therapeutic system to be localized in images of the treated region, and monitored.

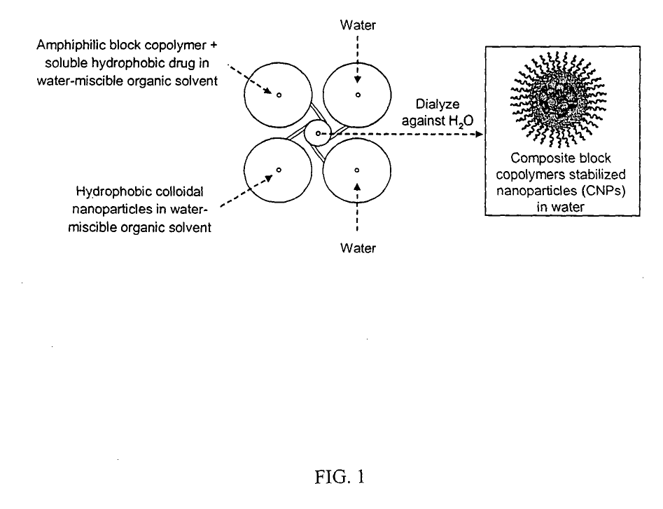

Flash nano-precipitation, as it relates to embodiments of the present invention, is a process for producing nanoparticles using rapid micromixing to effect mixing of highly supersaturated solutions of polymeric materials and hydrophobic materials such that their precipitation upon mixing, which is kinetically controlled, results in fine aggregations of hydrophobic compounds dispersed in a polymeric environment wherein the elements of the polymeric environment are, at least for preferable embodiments of the present invention, block copolymers that self-assemble into a colloidal structure having a hydrophobic core and a hydrophilic exterior. The structure is stable and behaves in aqueous environments as a nanoparticle (<1 micron). The process of preparing multicomponent (composite) nanoparticles using a multi-inlet vortex mixer is illustrated in FIG. 1. The process has been disclosed and described in Patent Application Publication No.: US 2004/0091546, International Publication No: WO 2006/014626, both of which are incorporated herein in their entirety by reference for all purposes.

Nanoparticles have become increasingly important in the development of new materials for enhanced drug delivery, and other materials for imaging applications (Adams, M. L. et al., J. Pharmaceutical Sciences 2003, 92:1343-1355; Portney et al., Analytical and Bioanalytical Chemistry 2006, 384:620-630). Drug carriers such as liposomal (Kim, S. Drugs 1993, 46:618-638), polymeric vesicle (Discher et al., Science 2002, 297, 967-973) and micellar dispersions (Allen et al., Colloids and Surfaces B—Biointerfaces 1999, 16:3-27; Kwon, Critical Reviews in Therapeutic Drug Carrier Systems 1998, 15:481-512) consisting of particles 50-400 nm in diameter have shown great promise, for example, in the formulation of anticancer therapeutics that would be highly insoluble in aqueous media absent their incorporation into a carrier. Such carriers, besides affording more potent drug delivery, also provide opportunities for selective tumor targeting.

More recently, inorganic nanoparticles, including quantum dots (Michalet et al, Science 2005, 307:538-544) gold nanospheres (West et al., Annual Review of Biomedical Engineering 2003, 5:285-292), nanoshells (Loo et al. Cancer Research and Treatment 2004, 3:33-40), and superparamagnetic metals (hornet et al., Journal of Materials Chemistry 2004, 14:2161-2175) have been explored for nanoparticle-based biomedical functions, such as tagging, medical imaging, sensing, and separation.

Despite extensive innovation over the last decade, there remains a need for imagible drug delivery modalities, especially modalities that can deliver highly toxic compounds in vivo, be they drugs or imaging agents. Polymeric nanoparticles in particular are a versatile medium for drug delivery, due to their enhanced drug loading capacity, biological stability, and extended in vivo circulation times (Kwon et al., Advanced Drug Delivery Reviews 1995, 16:295-309). Their utility for carrying potentially toxic imaging agents at the same time, however, remains virtually unexplored.

Polymeric nanoparticles that carry drugs and other agents encapsulated in their cores have evolved. Initially, research efforts focused on combining polymeric carriers of drugs with organic fluorescent dyes for particle visualization, without regard to the “encapsulation” of either the drug or the dye. Fluorescent nanoparticles have been prepared by binding water-soluble fluorophores to the surfaces of pre-formed nanoparticles (O'Reilly et al., Journal of Polymer Science Part A—Polymer Chemistry 2006, 44: 5203-5217) or, more commonly, by chemically tethering a fluorescent dye to the hydrophobic terminus of an amphiphilic block copolymer and then permitting the polymer to self-assemble into a particle (Luo, et al., Bioconjugate Chemistry 2002, 13:1259-1265). Organic dyes and fluorophores, however, require direct visualization, and so are generally practical only for in vitro applications such as nanoparticle cellular uptake and localization studies (Savic et al., Science 2003, 300:615-618).

Nanoparticles having a metallic core that adds contrast to images acquired by magnetic resonance imaging, for example, or computed X-ray tomography are, in principle, more suitable for in vivo biomedical applications (Bulte et al., NMR in Biomedicine 2004, 2004, 17: 484-499; Hainfeld et al., British Journal of Radiology 2006, 79:248-253). Typically, however, they are incompatible with body fluids because their surfaces are hydrophobic and they may also be incompatible because of toxicity. A number of coating strategies have been used to address these issues (Azzam et al., Langmuir 2007, 23:2126-2132; Kim et al., Langmuir 2007, 23: 2198-2202; Butterworth et al., Colloids and Surfaces A—Physicochemical and Engineering Aspects 2001, 179: 93-102; Gupta et al., Biomaterials 2005, 26: 3995-4021; Soo et al., Langmuir 2007, 23:4830-4836). Researchers have also functionalized the surfaces of such inorganic nanoparticles with receptor-specific peptides or protein ligands, allowing for targeted localization of the imaging particles (Paciotti et al., Drug Development Research 2006, 67:47-54; Zhang et al., Biomaterials 2002, 23:1553-1561; Zhou, et al., Biomaterials 2006, 27:2001-2008). Also, ligands (optionally together with drugs) can be covalently attached to the coating material instead of to the inorganic nanoparticle itself (Paciotti et al., Drug Delivery 2004, 11:169-183; Yu et al., Journal of Materials Chemistry 2004, 14: 2781-2786; Gupta et al., Biomaterials 2004, 25:3029-3040). Since the coating material is advantageously hydrophilic, however, the strategy of attaching hydrophobic moieties (e.g., drugs) to it is generally not practical.

A new process for manufacturing nanoparticles that encapsulate not only organic agents but, in addition, inorganic particulate nanostructures, has been described and claimed in a commonly assigned copending application. The process is described herein in the same detail as in the copending application. The process comprises the steps of
a. providing a hydrophobic organic compound dissolved in a solvent,
b. providing a hydrophobic inorganic nanoparticle dispersed in said solvent,
c. providing an amphiphilic polymer dissolved in said solvent, and
d. micromixing said solvent with an anti-solvent such that a composite nanoparticle forms.

The claimed process provides means for letting the organic compound, the inorganic nanoparticle or the amphiphilic polymer into the micromixing step of the process in a different solvent, or in the same solvent, by way of a different inlet. The inorganic nanoparticle may be surface-modified prior to step d above. It may also be functionalized to give the nanoparticle a hydrophilic surface. The organic compound may be electrostatically charged prior to step d, as may the inorganic nanoparticle.

The claimed process produces nanoparticles that comprise
a. a hydrophobic organic compound,
b. a hydrophobic inorganic nanoparticle, and
c. an amphiphilic polymer.

The composite nanoparticles do not flocculate in aqueous solvents.

The amphiphilic polymer may be selected from the group consisting of any copolymer, block copolymer, graft copolymer, comb-graft copolymer, and random copolymer that contains both hydrophobic and hydrophilic regions within the same copolymer.

The inorganic nanoparticle may be selected from the group consisting of magnetic, paramagnetic and superparamagnetic metals, and oxides thereof, or from the group consisting of gold, palladium and oxides thereof, or it may be a quantum dot. Additionally, certain compounds which happen to have upconverting phosphor properties may be exploited for their imaging capabilities. These include lanthanides, uranides, and transition metal ions. Preferable are the trivalent ionic lanthanide species, hosted in a semiconductor or bulk oxide, sulfide or fluoride lattice, or chelated with an organic compound.

The organic compound and the inorganic nanoparticle are encapsulated in a hydrophobic core region of the composite nanoparticle, and a hydrophilic shell surrounds the core. Encapsulated compounds and particles may be stabilized therein by means of steric hindrance, electrostatic charge stabilization or a combination thereof.

The composite nanoparticle may further comprise a targeting agent, which may be anchored on the external surface of the hydrophilic shell. The encapsulated organic compound or the inorganic nanoparticle may be releasable from the composite nanoparticle, and the released compound, the released nanoparticle, or both, may have targeting properties.

It is widely understood that, in contrast to molecules and the atoms of ionic crystals, such as sodium chloride, that dissolve in dilute solution and move about independently in (dilute) solution, nanostructures do not dissolve in a solvent into individual atoms or molecules as an ordinary crystal would. The atoms of a nanostructure are organized as a solid at the core. Solvents do not disrupt the core. Thus, nanoparticles disperse in solvents as particles, not as individual atoms or molecules. In part because of this, they attract one another readily, and aggregate readily.

To make a nanoparticulate carrier that is a composite of hydrophobic organic compounds and inorganic nanostructures, which carrier is to remain dispersed in an aqueous solvent, one is faced with finding a way of (1) stabilizing dispersions of nanostructures in a local environment that is loaded with hydrophobic organic and inorganic agents (Liu et al., International Journal of Cancer 2007, 120: 2527-2537; Fahmy et al., The AAPS Journal 2007, 9:E171-E180) and (2) stabilizing a population of the carriers as a dispersion in an aqueous solvent, bearing in mind that the carriers are themselves nanoparticles susceptible to aggregation.

The polymeric component of such a carrier suggests the possibility of stabilizing dispersions within the carrier sterically; that is, by utilizing the polymer to build barriers between nanoparticles in the dispersions. The advantages of such delivery systems, viz., high drug-loading capacity, reduced toxicity, protection of carried agents from the surrounding environment, targeting, localization and monitoring, and the possibility of tailoring the system's drug release kinetics (Kwon et al., 1998; Soppimath et al., Journal of Controlled Release 2001 70:1-20) are appreciated, but only a limited literature on the successful preparation of hybrid organic-inorganic nanoparticle formulations exists. For example, Gao and coworkers (Nasongkla et al., Nano Letters 2006 6:2427-2430) have described the preparation of composite poly(ethylene glycol)-block-poly(D,L-lactide) micelles encapsulating chemotherapeutic doxorubicin and superparamagnetic iron oxide nanoparticles. More recently, the successful preparation of antibody-conjugated poly(D,L-lactide-co-glycolide) nanoparticles incorporating doxorubicin and magnetic iron nanocrystals was reported by Yang et al. (Journal of Materials Chemistry 2007, Advance Article). In both cases, enhanced cancer cell affinity and improved magnetic resonance signals were reported in vitro. Unfortunately, the preparative techniques that were employed, namely solvent evaporation and emulsification processes, suffer from several disadvantages. First, they require the use of stabilizing surfactants and numerous purification stages to achieve only a low yield of uniformly sized nanoparticles. Additionally, hydrophobic components are relatively insoluble within the particles, so they cannot host hydrophobic components at high capacity (Shuai et al., Journal of Controlled Release 2004, 98:415-426). Lastly, these preparative processes do not allow the artisan to independently specify the amounts or kinds of individual components that the final nanoparticle will carry. Constraining the artisan are the unequal solubilities and miscibilities among the hydrophobic components, any nanostructures to be incorporated, and the hydrophobic domains of the stabilizing polymers. Furthermore, the processes do not ensure uniform distribution of actives within the nanoparticulate carriers.

The composite nanoparticles produced by the process described herein (and claimed in a commonly assigned co-pending application) carry organic and inorganic materials in nanoparticulate “capsules” whose outer surfaces are compatible with aqueous solvents and remain homogeneously disperse in them. In particular, such nanoparticles can carry an organic photosensitizer and an inorganic upconversion phosphor.

In photodynamic therapy, as previously noted, a photosensitizing compound must be delivered by one means or another to the proximity of a tumor, creating a photosensitized target. While the Applicants will not be bound by any mechanistic explanation of any embodiment of the invention, it is generally accepted that light of a specific wavelength can excite electrons in the molecules of a photosensitizing compound to a high energy state (higher, that is, than the compound's so-called “ground state”). If there is oxygen nearby, the oxygen can “couple” to the excited photosensitizer and take the photosensitizer's “excess” energy from it. The transferred energy tends to re-arrange the electrons in the coupled oxygen molecule in such a way as to create a so-called “singlet” oxygen. Singlet oxygen reacts aggressively with almost any biomolecule and disrupts it. Tumor cells inundated with singlet oxygen molecules soon die as a result.

The upconversion nanoparticles of the invention are conveniently solubilized in any aqueous vehicle that is compatible with cells and tissues in vivo. At the option of the user, the nanoparticles may be solubilized in a vehicle adapted for intravenous injection or injection into any other body cavity or body fluid, or (by way of example and not of limitation) in a vehicle adapted for aerosolization, lavage, or instillation. The nanoparticle-containing vehicle is administered in an amount and for a time sufficient to expose the target tissue of interest in the region of interest such that the tissue in the region is effectively photosensitized. The artisan will determine dosage amounts, forms and regimens depending upon the target tissue and region of interest, according to well-known protocols for administering other agents that bind thereto with like ligands. The artisan will select and control a light-source based on the light-absorbing and light-emitting properties of the selected photosensitizer, which properties are either known or readily ascertained, as are the transparency of the nanoparticle to impinging and emitted light, and the concentration of photosensitizer in the nanoparticle. In some embodiments, moreover, the nanoparticles are imaged in real-time or near real-time to guide the artisan in timing the exposure of the nanoparticles to the sensitizing light. In one embodiment, the light is applied immediately after unbound nanoparticles are cleared from the region of interest.

In one embodiment, the exposure to light is delayed after the nanoparticles are first administered. In one embodiment, the delay is less than about 15 minutes in length. In one embodiment, the delay is from about one hour in length to about 4 hours in length. In one embodiment 2 or more doses of nanoparticles are administered before the target region is first exposed to light. In one embodiment, the target region is first exposed to light when successive images of the target of interest show at least half-maximal occupancy of the region of interest by the nanoparticles. Other protocols may be appropriate depending upon the photosensitizer, upconversion material, region of interest, light source, choice of ligand used to “home” the nanoparticle on the target of interest, and other variables. At least with respect to the hydrophobic photosensitizers taught therein, U.S. Patent Application Publication 2004/0147501, incorporated herein in its entirety for all purposes, sets forth principles to guide the artisan in applying radiation to photosensitizer systems in the context of photodynamic therapy. The publication is also instructive regarding homing ligands, including ligands that promote penetration of photosensitizer systems into a tumor or tumor cell. The ligands may be attached to the terminal ends of the hydrophilic block of the stabilizing block copolymer used to form nanoparticles. The formulation may include 100% ligand functionalized hydrophilic block down to 1% functionalized hydrophilic block. The ligands may be incorporated prior to formation of the composite nanoparticle by for a post-formation derivitization of the hydrophilic block as described by Torchilin (Roby et al., European Journal of Pharmacology and Biopharmacology 2006, 62:235-240).

In describing the embodiments, the following meanings attach to the terms employed. Unless otherwise noted, all terms of art, notations, scientific terms or other terminology have the meaning commonly understood by persons of ordinary skill in the art to which the embodiments of the invention pertain, but may be defined herein for clarity or convenience.

As used herein, “a” or “an” means “at least one” or “one or more.”

The term “active agent” or “agent” refers herein to any chemical moiety or substance that has a desired behavior or activity. Non-limiting examples include elements, inorganic or organic ions, molecules, complexes, particles, crystals and radionuclides that may be “active” (without limitation) as pharmaceuticals, as contrast agents in imaging applications, as colorants, flavors, and fragrances, as sources or absorbers of energy, as linking or binding agents, or as toxins.

As used herein, a “nanoparticle” is any object of a size less than about 1 micron. It is not necessary that such a particle conform to this limit in all of its dimensions. Indeed, it is not even necessary that such a particle have dimensions in the conventional sense. Quantum dots, for example, may be referred to as “nanoparticles” herein. An “inorganic nanoparticle” as the term is used herein, generally refers to a particle comprising a metallic element, and an “organic nanoparticle” generally refers to a particle comprising a polymer (nanoparticles structured from elemental carbon are generally not regarded as “organic”). Particles comprising a polymer and at least one other material may be defined as “composite nanoparticles,” but the term as used herein generally refers to nanoparticles constructed of a polymer, an inorganic nanoparticle, and another component that is neither a polymer nor an inorganic nanoparticle. Preferably, the latter is an organic, hydrophobic small molecule.

As used herein, “dissolved” or “molecularly dissolved” molecules or atoms are homogenously distributed in a solvent and move about therein randomly and largely independently of one another. A substance that is “insoluble in an aqueous medium” dissolves in solutions that are physiologically relevant with respect to ionic strength, osmolality and pH only to the extent of 0.05 mg/ml or less. A solution that is “insoluble in pure water” dissolves in pure water only to the extent of 0.05 mg/ml or less. The criterion for “soluble” on the other hand is 1.0 mg/ml or more.

A “colloid” as used herein is analogous to a solution: both are systems of molecules, atoms or particles in a solvent. The particles of a colloidal system, however, because of their size (nanometers) or the distance between them (also nanometers), and their their solid cores, attract one another with sufficient force to make them tend to aggregate even when the only means of transport for the particles in the solvent is diffusion (the “diffusion-limited regime”). As used herein, the term “colloidal particles” refers to particles capable of forming a colloid. Although a “colloidal particle” is not itself a colloid but only a constituent of a colloid, the term “colloid” is often used to denote the particle itself. Thus, when the context so admits, the term “colloid” may refer herein to a particle. The term “colloidal dispersion” herein distinguishes colloids from true solutions on the one hand and from “suspensions” of larger particles that on the other hand. In the latter, the particles tend to “settle out” like sand stirred in water. A colloid, in contrast, tends to “flocculate” when large aggregates of particles form in the dispersion. The terms “colloidal dispersion” and “colloidal suspension” are often used interchangeably and may be so used herein. Instead of being “dissolved” in a solvent, the particles in a colloidal dispersion are said to be “solubilized” in the solvent. The solvent may be referred to as a “continuous phase” and, more colloquially, as the “surrounding environment.”

As used herein, the term “emulsion” is a dispersion of liquid droplets or liquid crystals in a liquid, wherein the droplets or crystals are generally larger than the particles in a colloidal system.

“Surfactants,” although they typically have hydrophilic and hydrophobic units, are distinguished from amphiphilic polymers herein. The size of a surfactant's hydrophilic unit (<1,000 MW) is too small to stabilize the composite nanoparticles as defined herein and a surfactant's hydrophobic unit (<750 MW) is too small to irreversibly anchor nanoparticles carried in the composite nanoparticle.

As used herein, a “polydisperse” colloid comprises particles that range in size. In a “narrowly polydisperse” colloid, the range is small.

As used herein, “organic compounds” encompass the entire domain of organic chemistry. Unless the context admits otherwise, however, organic compounds are) generally distinguished herein from polymers.

As used herein, a “hydrophobic moiety” is insoluble in aqueous solutions as defined above. The “moiety” may be, without limitation, a small molecule, a nanoparticle, a polymer or a region of a polymer.

Colloidal particles may be hydrophobic or hydrophilic. Colloids (i.e., colloidal dispersions) may also be referred to herein as “hydrophobic” or “hydrophilic.” A colloidal dispersion comprising an aqueous continuous phase with hydrophobic particles dispersed therein is referred to as a hydrophobic colloid or hydrophobic dispersion. Hydrophobic dispersions are thermodynamically unstable if the dispersion medium (or continuous phase) is aqueous. Conversely, a hydrophilic dispersion may be unstable if the dispersion medium is a non-polar solvent. Amphiphilic stabilizers may be incorporated into such dispersions to counter the instability. As used herein, an “amphiphilic stabilizer” is a compound having a molecular weight greater than about 500 grams/mole that has a hydrophilic region or domain and a hydrophobic region or domain. Preferably, the molecular weight is greater than about 1,000, or 1,500 or 2,000, and may be much higher, e.g., 25,000 or 50,000 grams/mole. Preferably, an amphiphilic stabilizer is a polymer, and more preferably a polymer or polymer system that provides both hydrophobic and hydrophilic domains to the colloid Block copolymers, graft copolymers, comb-graft copolymers, and random copolymers that contains both hydrophobic and hydrophilic regions within the same copolymer are useful.

As used herein, the term “mixing” may refer, when the context so admits, to “micromixing,” which has a particular meaning herein as set forth in detail below.

Conventionally, the term “anti-solvent” relates to a solvent which, when admixed with a solution comprising a second solvent, tends to cause the solute in the second solvent to precipitate. When admixed with a colloid, an anti-solvent may cause flocculation, in analogy with precipitation of a solute, but admixing a solvent containing dispersed nanoparticles and dissolved organic moieties with an anti-solvent may cause, instead of precipitation or flocculation, the self-assembly of particles of a different construction, as is described herein. The latter are distinguished herein from the “pre-formed” or “pre-existing” nanoparticles that may be incorporated therein. For clarity, the usual implication that an anti-solvent precipitates a solute, does not obtain herein.

As used herein, the term “surface-modification” refers to a process wherein reactive chemical groups on a surface, in particular the surface of a nanoparticle, are added to, removed from, or altered. The modified surface is sometimes referred to as having been “functionalized.”

As used herein, the term “encapsulation” relates to protecting constituents of the composite nanoparticle embodiments of the invention from reacting with or diffusing into the medium in which the composite nanoparticle is dispersed. Any such constituent is said to be “incorporated” in the nanoparticle as a “carried agent.” No distinct structural element is required to confer the protecting function. Similarly, the term “shell” relates herein to the protective function of a shell and not to any particular structure; materials “incorporated” into such nanoparticles. The shell of a nanoparticle may confer hydrophilicity or hydrophobicity on its nanoparticle, may be surface-modified, and may have adsorbed, bound or otherwise anchored to it, without limitation, molecules that interact (bind, react with, complex with) specifically with desired sites in or on materials, including without limitation natural or synthetic fibers, and plant or animal cells. Such molecules can include, without limitation, antibodies, receptor ligands, and any other means of linking a nanoparticle to a site of interest. Nanoparticles so modified are said to be “targeted” to the desired site, and the antibody, ligand, etc. may be referred to herein as a “homing” molecule or agent. The shell may be used to affix a “tag” (viz., a means that emits a detectable signal) to a nanoparticle to monitor the whereabouts, the integrity, etc. of the nanoparticle. Alternatively, such a tag may be incorporated into the nanoparticle. As used herein, a “capsule” or “shell” may also have the property of allowing encapsulated materials carried in the nanoparticle to be controllably released therefrom.

In some embodiments, the ligand, targeting agent or homing agent is specific for a receptor expressed on the surface of a tumor cell. Many tumors of the lung, for example, are called “neuroendocrine tumors” because they express receptors for hormones traditionally associated with the neuroendocrine system. Most non-small cell lung carcinomas, for example, express luteinizing hormone releasing hormone (“LHRH”) receptors. Accordingly, in some embodiments of the present invention, the ligand attached to the nanoparticle's surface is LHRH. In preferred embodiments, LHRH is tethered to about 25% of the PCL-b-PEG chains protecting the nanoparticle's surface.

The term “core,” as it relates to the nanoparticles referred to herein, is a solid-state material which, in its nanoparticle, is not susceptible to dissolution in the nanoparticle's dispersion medium.

The term “kinematic viscosity” as used herein refers to the tendency of a fluid to resist flowing (viscosity), factored by the fluid's density. It is the viscosity of a fluid divided by its density.

As used herein, the term “surface plasmon resonance” relates to a phenomenon detectable on metallic surfaces. While Applicants use the term in describing a measurement of use in some embodiments of the invention, the embodiments do not thereby rely on any mechanism put forth to explain the term. To better grasp the details of the method, however, the origins of the term are here briefly described. Metals have “free” electrons. They do not “belong” to any particular atom, but move about within the metal and also on its surface (as a so-called “plasma” or “electron gas”). Electromagnetic surface waves attend the movement. The waves, because they are “quantized,” have particle-like properties. The “particles” are called “plasmons.” Plasmons can interact with light and change its behavior in detectable ways. Under appropriate conditions, the interaction is “resonant.” That is, a plasmon can absorb the light's energy, producing what is appreciated by the observer as a “shadow.” These “shadows” are a manifestation of surface plasmon resonance.

The term “cancer” is a general term for more than 100 diseases that are characterized by the uncontrolled, abnormal growth of cells. Cancer cells can spread locally or can intravasate and spread via the bloodstream and lymphatic system to other parts of the body and form metastases. Cancer cells that spread are called “malignant.” The term “malignant” refers to having the properties of anaplasia, penetrance (into the vasculature) and metastasis, said of tumors. The term “tumor” refers to an abnormal mass of tissue that results from excessive cell division that is uncontrolled and progressive. It is also called a neoplasm. Tumors perform no useful body function. They may be either benign (not cancerous) or malignant.

“Infrared” (or “infrared light”) refers herein to electromagnetic radiation of a wavelength longer than visible light but shorter than that of radio waves. In preferred embodiments herein, “near infrared,” defined at wavelengths longer than about 750 nanometers and shorter than about 1,400 nanometers, is employed.

“Infra-red activable” as used herein refers to any chemical process or reaction whose activation is provided by infra-red light, either directly by means of absorption of the light by a reactant or indirectly by means of absorption by a chemical operably linked

As used herein, “active oxygen species” may be any chemical form of oxygen that is more chemically reactive than stable molecular oxygen (“triplet oxygen”). Such species include but are not limited to “singlet oxygen” (i.e., molecular oxygen in either of its two metastable states), and oxygen free radicals.

A “photosensitizer” renders the material it affects subject to the influence of radiant energy. In preferred embodiments of the instant invention, the term relates to agents which, when stimulated by light, react with oxygen in the affected material to produce active oxygen species.

As used herein, the term “upconverting (or ‘upconversion’) phosphor” relates to a material that absorbs radiant energy of wavelength longer than that of visible light and emits the absorbed energy as visible light. In preferred embodiments of the present invention, the energy for absorption is in the infra-red.

Flash nano-precipitation is a micromixing process comprising the steps of dissolving a hydrophobic organic compound in a compatible solvent, providing a polymer also dissolved in the solvent or in an aqueous solvent that is an anti-solvent to the organic compound, and rapidly micromixing the organic solution with the anti-solvent. The materials dissolved in the solvent(s), upon mixing in the anti-solvent, supersaturate the mixture and shortly precipitate into a population of uniformly sized nanoparticles. The kinetics of the process afford sufficient control to allow the artisan to mix hydrophobic organic compounds with amphiphilic polymers to produce nanoparticles of predictable size and stability (Johnson et al., Australian Journal of Chemistry 2003, 56: 1021-1024). The process has been disclosed and described in U.S. Patent Application Publication No. 2004/0091546 and in International Publication No.: WO 2006/014626, both of which are incorporated herein in their entirety by reference for all purposes.

The new process described here and also in a commonly assigned co-pending application, provides a method comprising the steps of dissolving a hydrophobic organic compound in a solvent, dispersing solid inorganic nanoparticles as a colloidal dispersion in that or another solvent, providing a polymer dissolved in that or another solvent (which may be an aqueous solvent that is an anti-solvent to the organic compound), and micromixing the organic solution, the dispersion and the anti-solvent such that polymeric nanoparticles are formed that retain, sterically stabilized therein, the hydrophobic organic compounds and the solid inorganic nanoparticles. Organics, including but not limited to organic fluorescent materials and therapeutic agents such as vitamins, anti-cancer agents, anti-bacterial agents, steroids, or analgesics may be incorporated into the composite nanoparticle. Solid inorganic nanoparticles including but not limited to imaging agents such as iron oxide nanoparticles, gold nanoparticles, gadolinium, and quantum dots may also be incorporated. Because the encapsulating nanoparticles of these embodiments are produced by means of flash nanoprecipitation, the use of surfactants to stabilize the nanoparticulate dispersions therein is unnecessary, uniformity of particle size is intrinsic to the process rather than a consequence of post-process purification, and the loading capacity for hydrophobic components is high.

In preferred embodiments of the present invention, an organic moiety incorporated is a photosensitizer, and a solid inorganic nanoparticle incorporated is an upconversion phosphor.

The process of preparing multicomponent composite nano-particles using a multi-inlet or multi-stream vortex mixer is illustrated in FIG. 1. The co-encapsulation of organic soluble molecules and inorganic colloidal nanostructures is illustrated. Alternatively, a confined impinging jet mixer as described in U.S. Patent Application Publication 2004/0091546 (incorporated herein in its entirety by reference for all purposes) can be employed.

Especially where unequal momentums of the organic and aqueous streams are advantageous, the multi-stream vortex mixer may be more suitable. Utilization of the multi-stream vortex mixer yields added flexibility in solvent selection, loading of multiple active agents and reduction of solvent to anti-solvent ratios. If two (or more) active agents are incompatible together in an otherwise convenient solvent, the two agents can be mixed from two separate solvent streams, and the velocity of each stream can be separately controlled. A constant flow rate can be provided by a syringe pump for each inlet tube using a Harvard Apparatus pump (model number 7023).

An exemplary but non-limiting multi-inlet vortex mixer, made of any rigid material, comprises a generally cylindrical mixing chamber 0.2333 inches in diameter and 0.0571 inches in height. The chamber is defined by a surrounding wall, a first cover or plate sealably disposed in orthogonal relation to the wall and, opposed thereto, a second sealable cover or plate. Four hollow cylindrical inlet tubes, each 0.0443 inches in diameter, penetrate the wall of the mixing chamber tangentially and, preferably, equidistantly, and are in fluid communication with the chamber. A hollow cylindrical outlet tube, 0.052 inches in diameter, has its long axis (approximately 0.5 inches in length) disposed in orthogonal relation to the inlet tubes. The outlet tube sealably penetrates one of the plates centrally and is in fluid communication with the chamber.

In some embodiments, a confined impinging jet mixer is suitable. A constant flow rate is provided by a syringe pump for each inlet tube using a Harvard Apparatus pump (model number 7023). At least one 100 ml glass syringe (SGE Inc.) is connected to each inlet tube. Two solvent streams of fluid are introduced into a mixing vessel through independent inlet tubes having a diameter, d, which can be between about 0.25 mm to about 6 mm but are between about 0.5 mm to about 1.5 mm in diameter for laboratory scale production. The solvent streams are impacted upon each other while being fed at a constant rate from the inlet tube into the mixing vessel. The mixing vessel is a cylindrical chamber with a hemispherical top. The diameter of the mixing vessel, D, is typically between 2.0 mm to about 5.0 mm, but preferably is between about 2.4 mm to about 4.8 mm. The mixing vessel also contains an outlet with a diameter, δ, that can be between about 0.5 mm to about 2.5 mm but is preferably between 1.0 mm to about 2.0 mm. The outlet of the mixer is connected to an 8-inch line of ⅛^{th}-inch tubing leading out for product collection.

The organic solutes, inorganic nanostructures and amphiphilic copolymers are dissolved, solubilized or dispersed, together or separately, in a water-miscible organic solvent including but not limited to tetrahydrofuran, dimethyl sulfoxide, or ethanol. In preferred embodiments, the inorganic nanostructures (generally sized between 1 nm to 700 nm) are “pre-formed” or “pre-existing” in the sense that they retain their discrete particulate nature when solubilized (dispersed) in the water-miscible organic phase and continue to retain it after being incorporated into the nanoparticulate product, even if that product simultaneously encapsulates organics. Pre-formed nanostructures are not formed during the nanoprecipitation process but beforehand.

Intense mixing (i.e., the mixing system operates at a Reynolds number >1600) of the organic solvent stream with water or a predominantly aqueous stream in the multi-inlet vortex mixer induces, in milliseconds, highly supersaturated mixtures (a solute “supersaturates” a solvent when the ratio of the concentration of the solute initially in the mixed streams in the mixing chamber to the concentration of the solute at equilibrium in the final solvent mixture is greater than 1). The artisan can readily measure the stream velocities of the inlet streams and the kinematic viscosity of each stream by means well known in the art, and can determine therefrom the Reynolds number for the system, defined as the sum of the stream flow rates times the average density of the fluids therein divided by the diameter of the inlet stream and divided by the average fluid viscosity of the streams. When solutes mixed under these conditions precipitate from a supersaturated state, nanoparticles of uniform size emerge. They are stable and remain dispersed as they leave the outlet tube. Actives captured within the particles also remain stable.

It is well within the skill of the artisan to “tune” the described mixing system to cause it to produce nanoparticles of a size between 1 nm and 10,000 nm (but preferably less than 1000 nm). A method (“dynamic light scattering”) for determining sizes of nanoparticles in the context of the relevant embodiments of the invention is set forth below. Thus, the artisan can select a size distribution that covers a fraction of this spectrum by tuning the system through solvent selection, choice of solute concentrations, stream velocities, conditions of temperature and pressure, and “time-scaling” as described in detail below.

The stability of the nanoparticles is also within the artisan's control, principally through the selection of polymers. In preferred embodiments, amphiphilic polymers or polymer systems are used. The relative sizes (molecular weights) of their hydrophilic and hydrophobic domains determines stability. The particles tend toward instability as hydrophobic domains are made smaller. As hydrophilic domains are made smaller, the particles may remain stable internally but, in dispersions, they will tend to aggregate and flocculate.

In polar liquids, charge stabilization (or “electrostatic stabilization”) by Coulombic repulsion is effective. In liquid dispersions, ionic groups can adsorb to the surface of a particle to form a charged layer. To maintain electroneutrality, an equal number of counterions with opposite charge will surround the particles and give rise to an overall charge-neutral double layer. The mutual repulsion of these double layers provides stability. Charge stabilization, however, is not effective in media of low dielectric constant (the vast majority of organic solvents and plasticizers) and thus steric stabilization is required to maintain the stability of dispersions of the particles. Steric stabilization of the colloid is achievable via attachment of macromolecules to the surfaces of the particles in the colloid. Although such attachment may be covalent in nature, it is typically adsorptive. That is, the macromolecule behaves as if “anchored” on the particle's surface but appropriate forces can displace the anchor-point to another site on the surface. Steric stabilization would appear to offer several distinct advantages over electrostatic stabilization, namely, relative insensitivity to the presence of electrolytes in the dispersion media, equal efficacy in both aqueous (polar) and nonaqueous (non-polar) environments, and equal efficacy at both high and low solids content.

In recent years, amphiphilic block copolymers have been demonstrated to be effective steric stabilizers of colloids. The amphiphilic nature of the block copolymer evidently allows one block to have a strong affinity for the hydrophobic materials in the core of the particle and serves to anchor the copolymer to the particle surface. The second block is more compatible with the dispersion media and provides a steric barrier towards particle aggregation and flocculation of the colloid.

In some embodiments, the present invention provides a simple process for producing polymer-encapsulated colloidal particles, each one of which itself comprises a stable colloidal dispersion. The encapsulating particle ranges in size, controllably, from about 25 to about 700 nm. Any colloidal particle dispersion of the present invention will have a distribution of particle sizes for a specific sample. The “size” is then denoted by one of the moments or averages of that distribution. This average is calculated by standard dynamic light scattering data analysis software such as CONTIN by Brookhaven Instruments, Long Island, N.Y. Alternatively, the size can be determined as the first cumulant of the distribution as again calculated using commercial dynamic light scattering software (Brookhaven Instruments, Long Island N.Y.). In the discussion that follows, if a single size is given it will be the first cumulant. And if the size distribution is given and an average size is quoted for the distribution it will refer to the light scattering average particle size described below. The process, moreover, affords the opportunity to control the degree of stability and thus the specific performance of products of the process.

In some embodiments, the invention provides a process for preparing composite nanoparticles from amphiphilic copolymers. The composite nanoparticles comprise inorganic particles encapsulated in the composite together with organic molecules as a colloidal dispersion that is capable of maintaining sufficient overall stability to accommodate a variety of post-processing manipulations. These manipulations include affixing targeting or “homing” molecules to the composite particles, and using the composite particles to transport molecules and particles to targets. Such molecules or particles may be incorporated in the composite particle or affixed, bound, or anchored to the surface thereof.

The process yields embodiments of the invention that are products characterized by narrow polydispersity and high loading capacity for one or more active agents stably incorporated in the particles. In one embodiment, the invention provides a process wherein a population of hydrophobic pre-existing nanoparticulate constructs, although surrounded by water, do not aggregate into a hydrophobic center, owing to the presence of an amphiphilic copolymer introduced by the mixing process described herein.

In some embodiments, the pre-existing particles are surface-modified before being mixed with polymer and organic moieties. A suitable but non-limiting method comprises bonding alkyl or aryl phosphonates to the surfaces of such particles as prescribed in commonly assigned U.S. Provisional Patent Application No. 60/951,113, filed on Jul. 20, 2007. A variety of surface-modifications are available, any of which may be selected, provided that the modification improves incorporation into the composite particle, the stability of the composite particle, the desired release properties or the desired targeting properties of the pre-existing particle (including compatibility at the target-site).

The pre-existing particle is dispersed in a solvent at a controlled temperature and pressure. An amphiphilic polymer is dissolved in a solvent capable of mixing with the solvent containing the pre-existing particle, but possessing different solubility characteristics for the amphiphilic polymer. The two solutions are then mixed at a controlled temperature and mixing velocity, causing selective precipitation of at least one portion of the amphiphilic polymer or polymer system while at least one other portion of the same polymer or polymer system remains soluble. In one embodiment, a product of the process comprises particles that have been functionalized by flash precipitation with an amphiphilic copolymer. In one embodiment, the copolymer is a block copolymer. Preferably, the average size of the functionalized particle is within 30% of its pre-process size if single hydrophobic particles are to be coated by the amphiphilic copolymer. The initial size of the functionalized particles can be between 50 nm and 50 μm. In a preferred embodiment, the ratio of pre-existing particle to amphiphilic copolymer is 1:1. If the desired composite nanoparticle is to include a plurality of smaller hydrophobic nanoparticles or hydrophobic nanostructures alone or in combination with hydrophobic soluble compounds, then the size of the resulting composite nanoparticle may be 60% larger to 400 times larger than an individual hydrophobic nanoparticle. Most significant to the stabilization of colloid in the nanoparticles in certain embodiments of the current invention is the attainment of millisecond micromixing, which is especially advantageous for the steric stabilization of sub-micron particles.

Examples of amphiphilic copolymers include but are not limited to block copolymers, graft copolymers, and random copolymers that contain both hydrophobic and hydrophilic regions within the same copolymer. The amphiphilic copolymer can be selected from several groups of copolymers including polystyrenes, polyethylene glycols, polyvinyl alcohols, polyvinylpyrrolidones, polyglutamic acids, hyaluronic acids, polyvinylpyrrolidones, polylysines, polyarginines, alginic acids, polylactides, polyethyleneimines, polyionenes, polyacrylic acids, and polyiminocarboxylates. Any biocompatible amphiphilic copolymer can be used. Preferably, the amphiphilic copolymer comprises diblock or triblock compositions containing at least one of the following: a polystyrene block, a polyethylene oxide block, a polybutylacrylate, a polyacrylic acid, polybutylmethacrylate block, or a polyethyleneoxide block.

Pre-existing particles can be comprised of biologically or organically active compounds or precursors including, but not limited to anti-inflammatories, anti-depressants, anti-oxidants, organic and inorganic pigments and dyes, proteins, water insoluble vitamins, fluorescent probes, agricultural actives or precursors, ceramics, latex, glass, or metal. Additionally, in certain embodiments, the present invention comprises simultaneously encapsulated hydrophobic and/or electrostatically charged pre-formed particles along with a dissolved hydrophobic active agent.

Some illustrative but non-limiting examples are provided herein for the better understanding of embodiments of the present invention. In those examples, particle size is characterized by dynamic light scattering analysis. The particle sizes are determined from the first cumulant fit of the dynamic light scattering correlation function (West et al., 2003.) The first cumulant fit Γ(q) is expressed as [1],

$$\frac{Γ ( q )}{{q}^{2}} = \frac{{\sum }_{k = 1}^{max} {n}_{k} {I}_{k} {D}_{k}}{{\sum }_{k = 1}^{max} {n}_{k} {I}_{k}} , \qquad ( 1 )$$

where Iₖ is the scattering intensity of particle k, nₖ is the number of particles of a given size, and Dₖ is the diffusion coefficient of particle k. The scattering wave vector q is given by

$$q = \frac{4 π n}{λ} sin ( \frac{θ}{2} ) , \qquad ( 2 )$$

where n is the refractive index of the solvent, λ is the wavelength of the incident light, θ is scattering angle. The first cumulant is related to the diffusion coefficient by,

$$\frac{Γ ( q )}{{q}^{2}} = {D}_{0} . \qquad ( 3 )$$

For dilute conditions, the Stokes-Einstein relation applies:

$${D}_{0} = \frac{kT}{6 πμ a} , \qquad ( 4 )$$

where μ is solvent viscosity. Combining Eqns. 1-4, we obtain an expression for the scattering intensity weighted radius of the particles, ā:

$$\frac{6 πμ \overline{a}}{kT} = \frac{{\sum }_{k = 1}^{max , ∞} {n}_{k} {I}_{k}}{{\sum }_{k = 1}^{max , ∞} {n}_{k} {I}_{k} \frac{kT}{6 πμ {a}_{k}}} \qquad ( 5 )$$

For the particles in the Rayleigh scattering range, where the size of the particles is much smaller than the wavelength of scattered light, the intensity of scattered light is proportional to the sixth power of the size for each particle.

This leads to the final expression for the diameter obtained by dynamic light scattering measurements:

$$\overline{a} ≡ {a}_{h [ 6 , 5 ]} = \frac{{\sum }_{k = 1}^{max , ∞} {n}_{k} {a}_{k}^{6}}{{\sum }_{k = 1}^{max , ∞} {n}_{k} {a}_{k}^{5}} . \qquad ( 6 )$$

Therefore, the a_{h[6,5]} moment of the size distribution is the appropriate moment to calculate from the simulations and to compare with the dynamic light scattering experiments.

Time-scaling. The process depends on tuning three time scales: 1) time to attain homogeneous mixing (τₘᵢₓ), 2) time for nucleation and growth of the hydrophobic actives (τ_{ng}), and 3) time of block copolymer self assembly (τₛₐ). The process has a characteristic mixing time in the range of milliseconds at a Reynolds number greater than 1600.

The mixing time is shorter than the timescale for nucleation and growth of dissolved organic solutes (τ_{ng}). By balancing the nucleation and growth times with the block copolymer assembly time, it is possible to block further particle growth and control nanoparticle size. Too rapid polymer self assembly consumes the stabilizer and results in uncontrolled growth, while too rapid nucleation and growth results in larger-than-desired particle sizes. The average nanoparticle size is thus controlled by the supersaturation levels and kinetics of aggregation of both the block copolymer and hydrophobic compounds.

### EXPERIMENTAL

All materials were purchased from Aldrich and, unless otherwise noted, used as received. Water, purified by reverse osmosis, ion-exchange, and filtration (Milli-Q water) was used for nanoparticlepreparation and dialysis.

### Example 1

### Synthesis of Poly(ethylene glycol-block-caprolactone) Block Copolymer

PEG-b-PCL block copolymers were synthesized by acid catalyzed ring-opening polymerization of ε-caprolactone (PCL) using monomethoxy poly(ethylene glycol) (mPEG) as an initiator according to published procedure (Shibasaki et al., Macromolecules 2000, 33:4316-4320). Dichloromethane and PCL were distilled from calcium hydride under reduced pressure shortly before use. Hydrochloric acid in diethyl ether was used as received. mPEG (5000 g/mol) was dissolved in tetrahydrofuran (THF), precipitated into cold hexane, and dried under vacuum. The polymer was further dried by azeotropic distillation of toluene under reduced pressure. To a solution of mPEG in dichloromethane was added PCL. Polymerization was catalyzed by addition of hydrochloric acid solution, and the reaction was carried out at room temperature for 24 h. The copolymer was precipitated into cold hexane, filtered, and dried at room temperature under reduced pressure. In THF at a concentration of 1 mg/ml, the copolymer absorbed no light anywhere in the UV-visible spectrum.

### Example 2

### Synthesis of Hydrophobic Gold Nanoparticles

Dodecanethiol modified gold nanoparticles (C₁₂—Au) were prepared by a two-phase reduction of hydrogen tetrachloroaurate (AuCl₄⁻) in the presence of dodecanethiol according to the method of Brust et al. (Journal of the Chemical Society—Chemical Communications 1994, 7:801-802. In brief, an aqueous solution of AuCl₄⁻ was mixed with a solution of tetraoctylammonium bromide in toluene. The mixture was vigorously stirred and the organic layer separated. Dodecanethiol was added to the organic phase and followed by the addition of aqueous sodium borohydride. The organic phase was separated and evaporated under vacuum. Gold nanoparticles were precipitated into cold ethanol, filtered and dried at room temperature under reduced pressure.

### Example 3

### Preparation of PEG-b-PCL Protected Gold Nanoparticles

A representative synthesis of block copolymer nanoparticles incorporating pre-formed nanostructures prepared via flash nano-precipitation is as follows. To a solution of PEG-b-PCL (5000-b-6000 g/mole) (55 mg) in THF (HPCL grade) (5 ml) was added dry C₁₂—Au nanoparticles (8.6 mg). The organic solution was fed (12 ml/min, stream 1), along with water (40 ml/min, streams 2-4), into a four-stream multi-inlet vortex mixer (FIG. 1) using two digitally controlled syringe pumps (Harvard Apparatus, PHD 2000 programmable, Holliston, Mass.), to yield a final solvent composition of 1:10 v/vol % THF:water. The concentrations of C₁₂—Au and PEG-b-PCL in final nanoparticle solution were 0.016 wt % and 0.1 wt %, respectively. Nanoparticles were dialyzed against Milli-Q water using a Spectra/Por® dialysis bag with MWCO of 6,000-8,000 (g/mole) (Spectrum Laboratories Inc., California, USA) and stored at room temperature.

### Example 4

### Characterization

Polymer molecular weights and polydispersity indices were measured by gel permeation chromatography (GPC) using a GPC unit (Waters Inc., Milford, Mass.) equipped with a series of Phenogel™ columns and a differential refractive index detector, calibrated with polystyrene standards (Polysciences Inc., Warrington, Pa.). High-resolution ¹H NMR spectra were obtained using a Varian Inova 400™ MHz spectrometer.

Nanoparticle size and size distributions were characterized via dynamic light scattering (Brookhaven Instruments, BI-200SM, Holtsville, N.Y.), consisting of double-pumped continuous NdYAG laser (Coherent Inc., wavelength 532 nm, 100 mW, Santa Clara, Calif.), and a photomultiplier with detection angle of 90°. The signal of the photomultiplier was analyzed by autocorrelation (ALV-Laser Vertriebsgesellschaft mbH, ALV-5000/E™, Langen, Germany), yielding the time-averaged scattered average particle size and polydispersity index (PDI). The particle size distribution was calculated using the ALV-5000/E™ software, from the decay-time distribution function with the assumption that the scattering particles behave as hard spheres (Bohren et al, Absorption and Scattering of Light by Small Particles, John Wiley, NY, 1983).

UV-visible absorbance spectra of nanoparticles were collected at room temperature using an Evolution 300™ spectrometer (Thermo Electron Inc., Madison, Wis.) in the wavelength range of 200-800 nm, with a resolution of 1 nm. Transmission electron microscopy (TEM) images were obtained on a JOEL 2010™ TEM microscope (Tokyo, Japan) working under an acceleration voltage of 200 kV. For the analysis, a drop of nanoparticles dispersed in water was deposited onto a carbon film supported by a copper grid and dried under reduced pressure. Observations were performed directly following grid preparation.

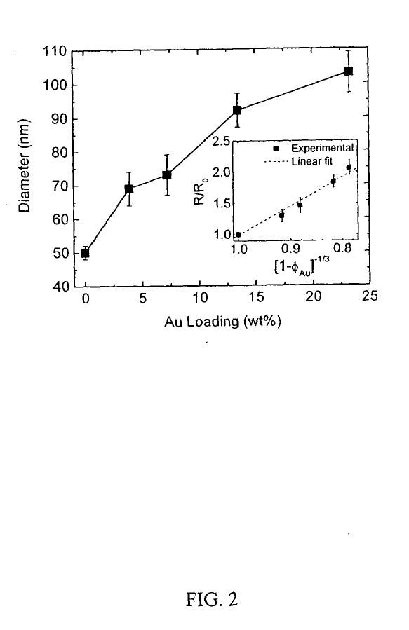

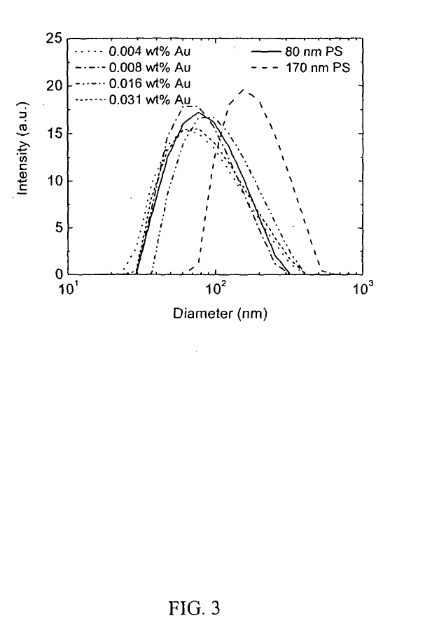

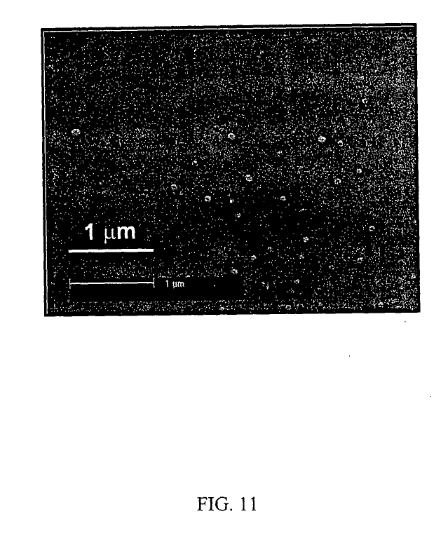

Mean particle diameters (hydrodynamic diameters) of PEG-b-PCL-protected Au particles prepared using the multi-inlet vortex mixer as a function of Au nano-particle loading are presented in FIG. 2. The error bars represent the standard deviation in measured diameters of several experimental runs generated at each condition. Nanoparticles were prepared at fixed block copolymer composition (0.1 wt % in the final solution) and Au loading is reported as solids weight percent (Au weight divided by Au and block copolymer weight). The mean size of unfilled polymer nanoparticles as prepared in the multi-inlet vortex mixer is 50±2 nm. Representative nanoparticles loaded at 7.2 wt % Au are shown in a scanning electron microscopic image in FIG. 11. The term ‘unfilled’ refers to nanoparticles prepared using only the block copolymer stabilizer, and which do not encapsulate any Au colloids. The average nanoparticle diameter is shown to increase with increasing Au concentration, reaching a value of 103±6 nm at a loading of 23 wt % Au. The inset of FIG. 2 shows the nanoparticle radius, R, normalized by the unfilled micelle radius, R₀, which scales with the gold colloid volume fraction (φ_{Au}) as R/R₀∝(1−φ_{Au})^{-1/3}. As elaborated below, the experimentally observed trend is predicted by a reaction model of colloid coagulation in the diffusion limited regime Fennel-Evans et al., Advances in Interfacial Engineering Series, 2^{nd} ed. 1999, 417-424. The corresponding particle size distributions, shown in FIG. 3, remain narrow, with PDI values less than 0.25±0.02 obtained in all cases. For reference, particle size distributions of polystyrene calibration standards of similar sizes (80 nm and 170 nm) are also shown in FIG. 3, with measured PDI values of 0.17±0.03 and 0.13±0.03, respectively.

Since no post-synthesis purification of nanoparticle solutions was performed, no material losses are associated with the particle preparation process and high volumetric productivity is achieved. Typical precipitation processes operate at concentrations below 0.05 mg/ml (Kim et al., Langmuir 2007, 23:2198-2202) of the block copolymer stabilizer and often require post processing purification for the removal of macroscopic aggregates, resulting in significant material losses and reduced colloid loadings, on average less than 10 wt % with respect to the block copolymer (Nasongkla et al., 2006). Using the multi-inlet vortex mixer, nanoparticles with Au loadings of greater than 20 wt % have been prepared at block copolymer concentrations in the range of 1.0-4.0 mg/mL, demonstrating both enhanced nanoparticle loading capacity and improved volumetric productivity.

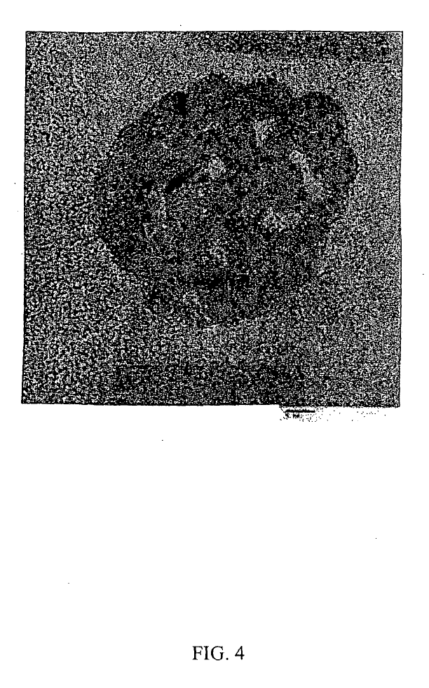

A representative TEM micrograph of PEG-b-PCL-protected Au nanoparticles is shown in FIG. 4. Contrast in the TEM image is provided only by the Au, as the block copolymer is unstained. Individual Au monomers, approximately 5 nm in diameter, are clearly visible. The random, close packing of the Au monomers within the particle core is evident. For a representative Au loading of 23.3 wt %, nearly spherical particles with a mean diameter of 103±6 nm, as determined by dynamic light scattering, are produced. Particle size and size distributions, as determined by dynamic light scattering, are in good agreement with TEM and SEM observations.

### Example 5

### Simultaneous Loading

The flash nanoprecipitation technology was used to simultaneously load hydrophobic organic actives and inorganic colloids for integrated drug delivery and imaging applications. The vitamin A precursor β-carotene was selected as a model hydrophobic compound and encapsulated, in conjunction with Au, within the cores of PEG-b-PCL nanoparticles using the multi-inlet vortex mixer as described. Using ratios of β-carotene:Au:block-copolymer of 30.5:5.0:64.5 wt % (fractional weight of β-carotene, Au, and block copolymer with respect to total solids mass), composite nanoparticles approximately 80 nm in diameter were prepared. To confirm capture of components within the nanoparticle interiors, an as-prepared nanoparticle solution was filtered through a 10K OMEGA™ nanoseparation centrifuge filter membrane (Pall Corporation, East Hills, N.Y.), which allows for the retention of composite nanoparticles on the membrane surface and permits passage of free β-carotene and unprotected Au colloidal particles.

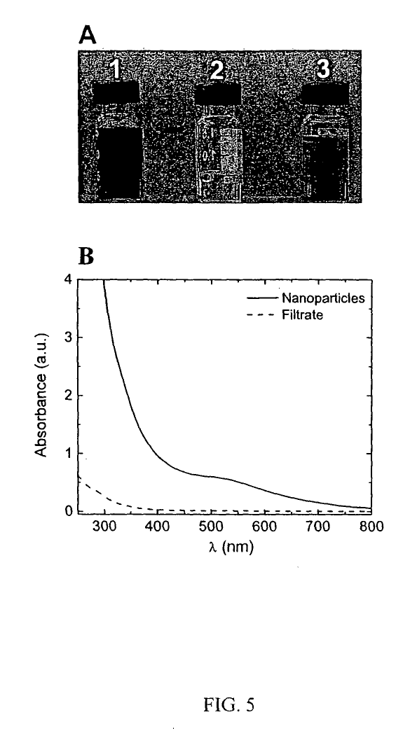

The encapsulation of Au monomers (9.4 wt %) and β-carotene (30.2 wt %) within composite nanoparticles prepared by flash nanoprecipitation was first examined visually as shown in FIG. 5A. The composite nanoparticle solution prior to filtration was deep red (vial 1), whereas the filtrate (after filtration via a 10,000 MW ultrafiltration membrane) was clear (vial 2). Complete capture of Au and β-carotene is indicated by transparency of the supernatant (vial 2) when compared to a solution of non-protected Au colloid suspended in THF filtered via the same membrane (vial 3). Total recovery of unprotected Au colloid through the nanoseparation filter (vial 3) was confirmed independently via UV-visible absorbance measurements at 520 nm, where Au colloids of this size exhibit a maximum in the absorbance spectrum. Transfer of β-carotene through the filter was expected based on the molecular weight of the molecule (536.9 g/mol).

Corresponding UV-visible absorption spectra of the composite nanoparticle solution and filtrate are shown in FIG. 5B. A distinct absorbance peak at 520 nm resulting from the presence of Au colloids is seen in the spectrum of the composite nanoparticle solution, whereas this peak is essentially undetected in the solution following filtration. Quantification of Au concentration in the composite nanoparticle and filtrate solutions was made based on calibrated measurements of absorbance values at 520 nm. The UV-visible spectrum of β-carotene does not interfere with that of Au in the wavelength range of 400-800 nm, and thus the absorbance value at 520 nm can be utilized to calculate Au concentration in the composite nanoparticle formulation. Based on this calibration, an Au encapsulation efficiency in excess of 99.5 weight percent was estimated.

The quantification of β-carotene composite nanoparticle loading is complicated by the overlap in absorption spectra of the two components in the UV region, where solutions of β-carotene exhibit an absorbance maximum at 290 nm. As such, we have alternatively prepared PEG-b-PCL nanoparticles in which β-carotene is independently encapsulated. Nanoparticles were isolated as previously described, and the concentration of free β-carotene in the filtrate measured. Based on UV calibration at 290 nm, a β-carotene concentration of approximately 0.05 mg/mL was estimated. This concentration corresponds to the solubility limit of β-carotene in the final solvent composition of 1:10 v/v % THF:H₂O. Thus, all β-carotene in excess of the solubility limit was incorporated within the nanoparticles. Because nanoparticle loading relies on compound solubility, the encapsulation efficiency of organic molecules will remain unaffected in multiple component formulations.

While the Applicants will not be bound by any particular theory as to the mechanisms by which any embodiment of the invention works, it is believed that the ability of this technology to provide quantitative homogeneous incorporation of actives arises from the very high level of supersaturation of all components, leading to rapid aggregation and controlled adsorption of the stabilizing polymer on the composite nanoparticle surface (Brick et al., Langmuir 2003, 19:6367-6380). The significant advantage of the process is that component loadings can be accurately specified a priori, in contrast to slow, quasi-equilibrium formation processes which lead to unequal incorporation of individual components depending on their solubilities. In our process, drug and imaging agent loadings can be optimized independently and subsequently formulated into a single multifunctional delivery vehicle.

### Example 6

### Stability

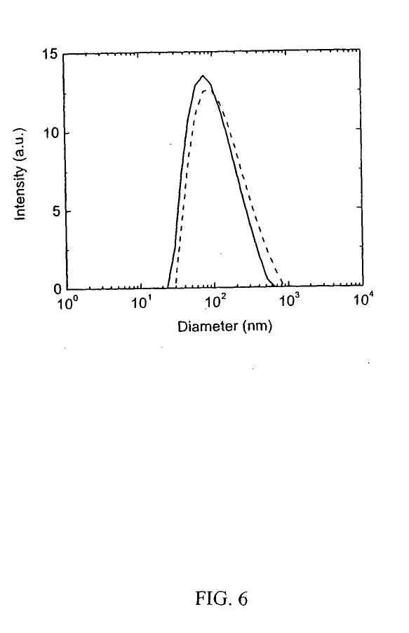

The extended stability of these particles in the presence of physiological salt concentrations was also investigated. Particle size distributions, determined by dynamic light scattering, of PEG-b-PCL (5,000-b-6,000 g/mol) nanoparticles encapsulating Au (9.4 wt %) and β-carotene (30.2 wt %) were stable over time. FIG. 6 shows particle size distributions of PEG-b-PCL-protected β-carotene/Au composite nanoparticles immediately after preparation and dialysis and after one month of storage in 155 mM saline at room temperature. The mean particle diameter and size distribution of the unfiltered solutions increased to a minor extent from approximately 85 nm to 100 nm over this time, indicating particle stability in aqueous environments for extended time periods. The slight increase in particle diameter arises from Ostwald ripening inherent in all nanometer scale particles (Liu et al., Physical Review Letters 2007, 98(3))

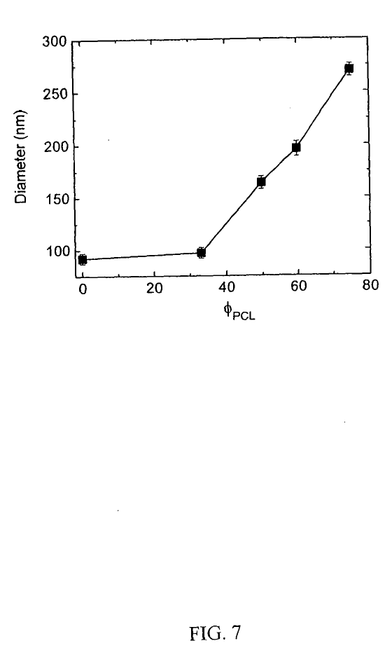

Nanoparticles in the size range of 100-300 nm are specifically of interest, as they have been exploited for passive delivery of anti-cancer agents to solid tumor vasculature, where defective vascular architecture and impaired lymphatic drainage allow for improved particle uptake and localization through the enhanced permeation and retention (EPR) effect (Duncan et al., Annals of Oncology 1998, 9: 39). We demonstrate the ability to control the size of composite nanoparticles within the above specified range in a predictable fashion in FIG. 7.

PEG-b-PCL-protected Au composite nanoparticles in which the particle size was ‘tuned’ through addition of an inert component, homopolymer PCL (3,200 g/mol), were prepared. For fixed colloid concentration (0.016 wt % in solution), composite nanoparticle size in the range of 75-275 nm is shown to be a nearly linear function of homopolymer volume fraction (φ_{PCL}) for PCL loadings above 33 vol %. While Applicants will not be bound by any mechanistic explanation of why any embodiment of the invention works, the relatively constant nanoparticle size with PCL addition for volume fractions below this value is speculated to result from initial filling of the interstitial voids created by the random packing of Au monomers in the nanoparticle core, estimated at approximately 37 vol % (Torquato et al., Physical Review Letters 2000, 84:2064-2067). The dense, random nature of monomer packing is supported by TEM imaging as shown in FIG. 4. PCL addition beyond this point contributes to increasing particle diameters. Nanoparticle size and active loading can thus be specified independently of one another, yielding a highly flexible nanoparticle formation platform.

### Example 7

### Prediction of Encapsulated Colloidal Particle Number and Nanoparticle Size

In FIG. 2, the size of polymer-protected Au nanoparticles was shown to be a function of colloidal particle concentration. In this Example, we illustrate that this behavior is well predicted using a simple representation of colloid self-assembly in the diffusion limited regime, as outlined by Fennell-Evans and Wennerstrom. In this model, a system of spherical particles each of uniform radius R undergoing Brownian motion is considered. The spheres are assumed to interact according to a square well potential of infinite energy with an interaction distance of 2 R. At steady state and in the diffusion limited regime, the rate constant for colloid association is shown to be independent of aggregate size and can be used universally to ascertain the kinetics of aggregation, yielding the following general solution to the aggregation process:

$$[ {P}_{N} ] = {[ P ]}_{0}^{tot} {( \frac{t}{τ} )}^{N - 1} {( 1 + \frac{t}{τ} )}^{- N - 1} , \qquad ( 1 )$$

where P_{N} is the concentration of particles each composed of N monomers, [P]₀^{tot} is the monomer concentration at t=0, and τ=2/(k[P]₀^{tot}), where k is the universal rate constant given by:

$$k ≡ \frac{8}{3} \frac{{k}_{B} T}{μ} \qquad ( 2 )$$

for which, k_{B} is the Boltzmann constant, T is the solution temperature, and μ is the solvent viscosity.

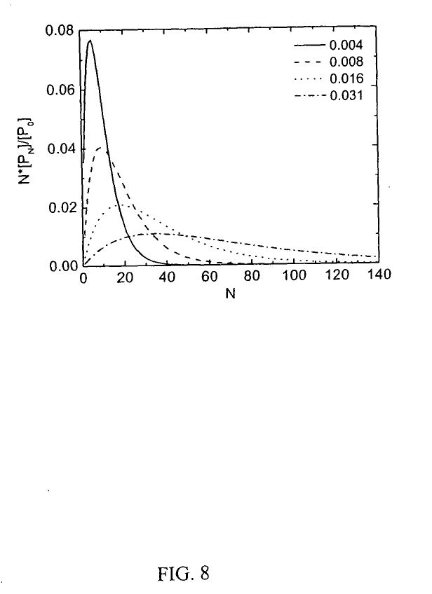

In the multi-inlet vortex mixer, rapid micromixing in the range of milliseconds is attained, yielding a homogeneous system in which colloid aggregation and block copolymer precipitation occur in the diffusion limited regime. In this manner, colloid aggregation persists until block copolymer deposition on the assembly surface limits further coagulation. Thus, the time allowed for colloid assembly will be dictated by the sum of the characteristic mixing time in the multi-inlet vortex mixer and the block copolymer induction time. In the case of PEG-b-PCL, the copolymer self assembly time is estimated based on a value for comparable molecular weight poly(ethylene glycol)-b-poly(styrene) (1,000-b-3,000 g/mol) block copolymers as reported in literature, where the induction time is approximated to be 37 ms (Johnson et al., 2003). Accounting for an estimated mixing time of 3 ms (Liu et al., Chemical Engineering Research 2007) in the multi-inlet vortex mixer, snapshots of the particle size distributions at a time of 40 ms are calculated using Eq. 1 for varying colloid concentrations (wt % in solution). The results of these calculations are shown in FIG. 8. For a given initial monomer concentration [P₀], the final distribution of aggregates, each composed of N monomers, at an assembly time of 40 ms is calculated. The normalized fraction of monomers participating in an aggregate, N*[P_{N}]/[P₀], is shown to reach a maximum for each colloid concentration studied, with a shift towards larger maximum values as the colloid concentration is increased. The model additionally predicts a corresponding increase in cluster distribution dispersity as the colloid loading is increased. This trend is supported experimentally, as evidenced by the slightly increasing PDI values of PEG-b-PCL protected Au nanoparticles with increasing Au content shown in FIG. 3.

Model predictions of cluster sizes calculated at t=40 ms were compared to particle diameter values, as determined by dynamic light scattering, for PEG-b-PCL protected Au nanoparticles prepared in the multi-inlet vortex mixer. For particles in the Rayleigh scattering range, the intensity of scattered light is proportional to the sixth power of the size for each particle (Bohren et al., 1983). This leads to the following expression for particle radius as obtained by dynamic light scattering measurements:

$$\overline{R} ≡ {R}_{6 - 5} = \frac{{\sum }_{N = 1}^{max , ∞} {n}_{N} {R}_{N}^{6}}{{\sum }_{N = 1}^{max , ∞} {n}_{N} {R}_{N}^{5}} \qquad ( 3 )$$

where n_{N} is the number of particles of a given radius R_{N}. For a given colloid concentration, particle size can be calculated analytically using Eq. 3 in conjunction with the particle size distributions, [P_{N}], calculated from Eq. 1. Calculation of nanoparticle core volume was made assuming each C₁₂—Au monomer occupies a radius of 4 nm (2.5 nm for Au core and 1.5 nm for C₁₂ extended chain length). Additionally, packing of the monomers within the nanoparticle core is assumed to be close packed and random in nature, occupying a volume fraction of 0.63. To account for the PEG-b-PCL stabilizing copolymer, the diameter of unloaded PEG-b-PCL nanoparticles prepared using the multi-inlet vortex mixer (50 nm) was added to the cluster diameters computed through the model.

TABLE 1
Calculated vs. experimental size of block copolymer protected
Au NPs prepared via Flash Nanoprecipitation.

| | | | Calculated average |
|---|---|---|---|
| | Calculated average | | number of Au monomers |
| | diameter (D₆₋₅) of | Experimental diameter | (N₆₋₅) in PEG-b-PCL |
| | PEG-b-PCL protected | of PEG-b-PCL protected | protected Au NPs with σ |
| Au | Au NPs with σ in value | Au NPs as determined | in value reported as |
| concentration | reported uncertainty | by DLS | uncertainty |
| 0.004 wt % | 81 ± 3 nm | 69 ± 5 nm | 38 ± 2 |
| 0.008 wt % | 89 ± 3 nm | 73 ± 6 nm | 70 ± 3 |
| 0.016 wt % | 98 ± 3 nm | 92 ± 5 nm | 109 ± 5 |
| 0.031 wt % | 105 ± 2 nm | 103 ± 6 nm | 126 ± 5 |

Au concentration in column one is reported as weight fraction of Au in solution. The standard deviations (σ) in calculated and experimentally determined values, rounded to the nearest integer, are reported as error.

Calculated cluster diameters as a function of colloid concentration are reported in Table 1 (column 2). The standard deviation in particle size calculated from simulations at each Au concentration is reported as the uncertainties. When compared to experimentally determined particle diameters, as obtained by dynamic light scattering (column 3), the results show that particle size was well predicted using this simple model of colloid aggregation. The model also allows for the prediction of colloid number density within the nanoparticle core. The intensity averaged particle aggregation number, N₆₋₅, was similarly calculated according to Eq. 3 and the results shown in Table 1 (column 4). The average aggregation number increased with increasing colloid loading, reaching a 126 (σ=5) for the highest Au concentration investigated. The estimated colloid loading density for this Au composition is well supported by TEM imaging for a similarly prepared sample as shown in FIG. 4. This characterization thus allows for accurate a priori determination of particle size and colloid loading based solely on process inputs, permitting incorporation of multiple inorganic colloidal components at independently specified concentrations.

### Example 8

### Properties of Self Assembled Nanoparticles

Physical properties of colloidal particles are expected to be preserved upon incorporation within the nanoparticle cores. The case of gold colloid encapsulation shown here is particularly interesting owing to the electronic behavior of nanometer sized gold crystals. Gold nanoparticles exhibit localized collective oscillation of surface conduction electrons, leading to distinctive surface plasmon resonance peaks in the UV-visible region (Terrill et al., Journal of the American Chemical Society 1995, 117:12537-12548). Since the surface plasmon resonance frequency of a particular sample of gold colloid depends strongly on the size, shape, dielectric properties, and aggregation state of the nanoparticles (Link et al., 2000), measuring the phenomenon in encapsulated forms of the colloid is useful in the engineering of gold-containing nanoparticles.

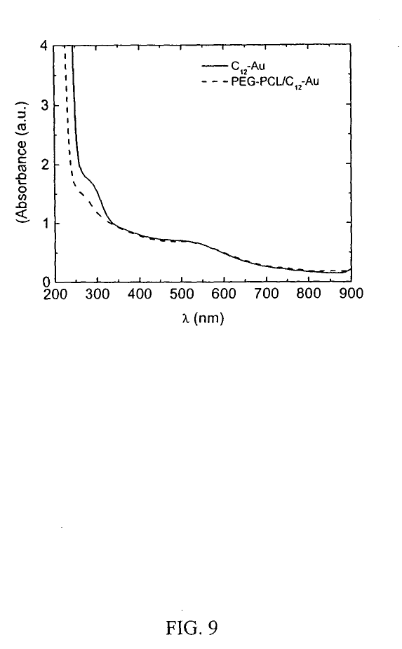

Encapsulation of Au particles within a block copolymer shell using the multi-inlet vortex mixer was shown to preserve the metallic properties of isolated Au nanoparticles. Although Applicants will not be bound by any theory seeking to explain why embodiments of the invention work, it is thought that when Au particles are in close proximity, they are able to interact electromagnetically, primarily through a dipole-dipole coupling mechanism. This mechanism broadens and red shifts the plasmon resonance bands (Link et al., 2000). FIG. 9 shows the recorded absorbance spectra for dispersions of C₁₂—Au in THF and PEG-b-PCL protected C₁₂—Au nanoparticles in a THF:water mixture (1:10 v/vol %). The peak in the extinction spectra, lying at approximately 520 nm, remains unaltered in the nanoparticle assembly, suggesting no overlap in the electronic structure of neighboring Au particles has occurred. The dodecane capping layer dictates the properties surrounding the gold nanoparticle (medium dielectric constant and refractive index), and its thickness, estimated between 1-2 nm (Terrill, et al., 1995), controls the separation distance between neighboring Au monomers, maintaining the particles in an electronically independent state. The inter-particle separation distance, and thus electronic properties of the aggregate, can thus be precisely controlled through selection of an appropriate capping ligand.

We have utilized this capacity for control over inter-particle distance to generate composite fluorophore-gold assemblies, in which enhanced fluorescence from the organic dye in the nanoparticle assembly is observed. This system is expected to provide a photostable imaging platform with the capacity for particle size control, multi-modal imaging, and reduced toxicity effects.

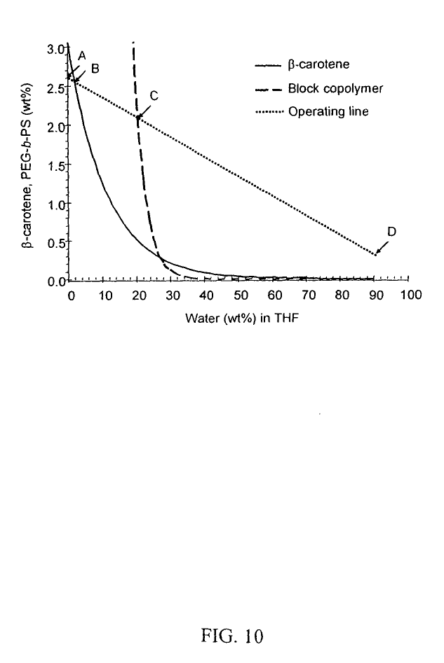

Finally, we want to place the process of flash nanoprecipitation we have used here in context with other block copolymer-based nanoparticle formation processes described previously. There are fundamental thermodynamic constraints which limit the ability of processes used by previous researchers (Nasongkla et al., 2006; Yang et al., 2007) to produce multifunctional nanoparticles at high loadings and with controlled particle size. Those limitations can be summarized in FIG. 10, which shows the precipitation concentrations, or solubility boundaries, for two components as a function of anti-solvent addition. Previous researchers have slowly added anti-solvent to initially soluble solutions of block copolymers and imaging agents or drugs (Allen et al., 2000; Kim et al., 2001). FIG. 10 displays the solubility of the organic active β-carotene and the solubility (critical micelle concentration) of a block copolymer stabilizer (poly(ethylene glycol)-block-polystyrene) as a function of THF content at 35° C. (Johnson, PhD Thesis, Princeton University, 2003). While the solubility data shown in FIG. 10 is specific to PEG-b-PS stabilized β-carotene nanoparticles, as detailed in previous work (Johnson et al., 2003), the operating line shown can be generally applied to describe the flash nanoprecipitation process. The method of slow anti-solvent addition involves traversing the operating line from the initially pure solvent condition (designated A) in which all components are soluble, to the intersection with the solubility curve for β-carotene (designated B) at 2.5 wt % water in THF. At this point β-carotene will begin precipitating. The stabilizing polymer does not start aggregating on the particle surface until the water concentration reaches 23 wt % (designated C). But at this point over 70% of the β-carotene has precipitated as unprotected crystals. Without subscribing to any theory of why embodiments of the invention work, it is thought, in the case of fast mixing, as achieved by the flash nanoprecipitation process (Johnson et al., AICHE Journal 2003, 49:2264-2282), such high levels of supersaturation are produced—in milliseconds—that, at the final solvent composition (designated D), all species aggregate by a diffusion limited, non-specific process. The composition of the resulting nanoparticles reflects the stoichiometry of the feeds and no unincorporated material is produced. In this manner, block copolymer nanoparticles at high drug loadings (2.6 wt % PEG-b-PS to 2.6 wt % β-carotene for the case shown) are easily prepared (Johnson et al., 2003).

### Example 9

### Photodynamic Therapy for Malignant Mesothelioma

Six to twelve week old, female BALB/cJ (AB12 tumor host) mice, obtained pathogen-free from Taconic Farms, were used for all of the experiments. Animals were kept in conventional conditions in micro-isolator cages in a laminar flow unit under ambient light with full access to food and water during experiments.

AB12, murine malignant pleural mesothelioma (MPM) was utilized for tumor inoculation. Cells were maintained in complete DMEM/F12 culture media in a 37° C. humidified 5% CO₂ incubator.

Up-conversion nanophosphor particles of yttrium and gadolinium (Gd) oxide co-doped with europium (Eu), erbium (Er), thulium (Tm), and ytterbium (Yb) ions were prepared using the flame synthesis method with vapor phase precursors. This method provides the advantages of high rate synthesis, small nano-size (<200 nm), non-agglomeration, and short time operation on the particle synthesis.

The vapor phase precursor was generated from the vaporization of Y(TMHD)₃, Er(TMHD), Tm(TMHD)₃, Yb(TMHD)₃, and Eu(TMHD)₃ salts at high temperatures. The precursors were housed in a preheated chamber, so that they could vaporize and produce a homogeneous mixture. The precursor concentration was controlled by the dilution of either argon or nitrogen gas for different particle sizes. The chamber temperature was controlled by an electrical heater with a temperature accuracy of 10° C.

The precursor was mixed with the co-flow streams of hydrocarbon fuels (hydrogen and methane) and air or oxygen. The fuel-air mixture was ignited to form a diffusion flame above the precursor nozzle. The precursor was decomposed and oxidized in the high temperature flame zone. The formed rare-earth particles collided with each other and resulted in large particles downstream. In order to suppress the particle growth and to control the particle size, a cold nitrogen gas was introduced to quench particle agglomeration. The synthesized particles were collected either by a filter or an electrostatic collector.

The method for thin layer SiO₂ coating was accomplished as follows: 3-aminopropyl trimethoxysilane (APTMS) was used as the organic precursor for coating. APTMS was preheated in a glass bath to vaporize (190° C.). The vapor of APTMS was carried by the nitrogen or argon inert gas to mix with the dried nanoparticles carried by another inert gas stream. A heater was used downstream of the mixing region to raise the temperature up to 250° C. At high temperatures (not so high as to damage the NH₂ group), APTMS reacts on the surface of nanoparticles and forms a saturated thin layer (a few nanometers) of silica. The structure and optical properties of the coated particles were collected by filters and analyzed by TEM and fluorescence spectrometry.

Seventy balb/C mice were inoculated with 5×10⁵ AB12, malignant mesothelioma cells via subcutaneous flank injection. The subsequent tumors were measured using digital calipers three times per week until the tumors reached ˜200-300 mm³ at which time the mice were given their first treatment. Mice were divided into five groups. The groups were as follows: i. Control, ii. Infrared light-Nanophosphor-Photosensitizer (IR-NP-PS), iii. IR-NP, iv. IR-PS, v. IR alone, vi. NP alone, and vii. PS alone. Groups ii, iii, and vi were administered intra-tumoral injections with 100 μl of 25 mg/ml nanophosphor suspension in saline at 72 and 48 h prior to only the first treatment. Groups ii, iv, and vii were administered weekly intra-peritoneal (i.p.) injections of 10 mg/kg Photofrin™, twenty-four hours before first treatment of each week.

For this procedure mice were anesthetized with i.p. ketamine and xylazine, 80 mg/kg and 12 mg/kg, respectively. A maximum dose of 1.0 W of 980 nm light (BW Tek) at the treatment surface was measured using an IR thermopile detector (Coherent, Inc) for a total dose of 28.3 J/cm². Pulsed light delivery method of 1 second ON, 2 seconds OFF for 1.5 hours was utilized to minimize damage of healthy tissue due to heat from the infrared light by allowing the tissue to dissipate the heat between delivery cycles. Treatments were administered bi-weekly until tumors completely regressed or showed no regression and reached tumor endpoint. Over the course of the entire treatment program tumors were measured three times weekly and mice were monitored for signs of morbidity for 60 days or until tumors reached endpoint of 500 mm³.

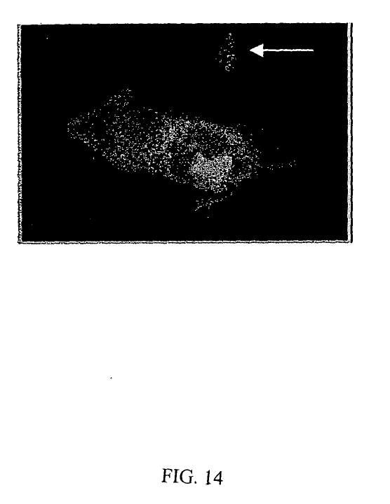

Characterization of the Si-coated nanophosphor using TEM and fluorescent spectrometry yielded ˜100 nm particles with an absorption band at 980 nm and two-photon emission spectra of 540 and 660 nm as can be seen in FIG. 14.

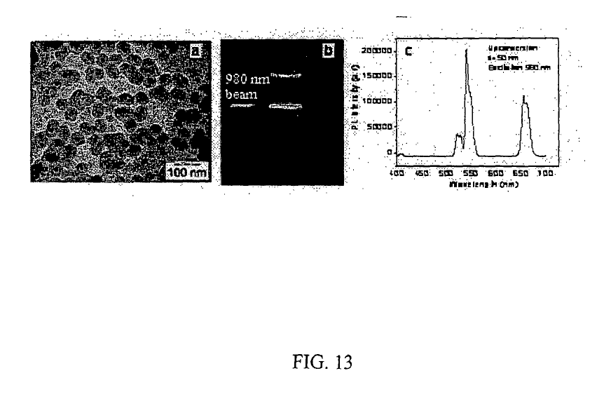

This in vivo study showed significant external PDT-mediated tumor regression and eradication of a stably growing murine mesothelioma. Applicants will not be bound by any mechanistic explanations, but the generally stated mechanism for activation of photosensitizer of the type used in this example is described as infrared light conversion to two-photon visible emissions by up-converting nanophosphors. FIG. 13 depicts a Balb/c mouse undergoing PDT. The nanophosphor was injected intra-tumorally prior to the first light delivery and is being activated by 980 nm light.

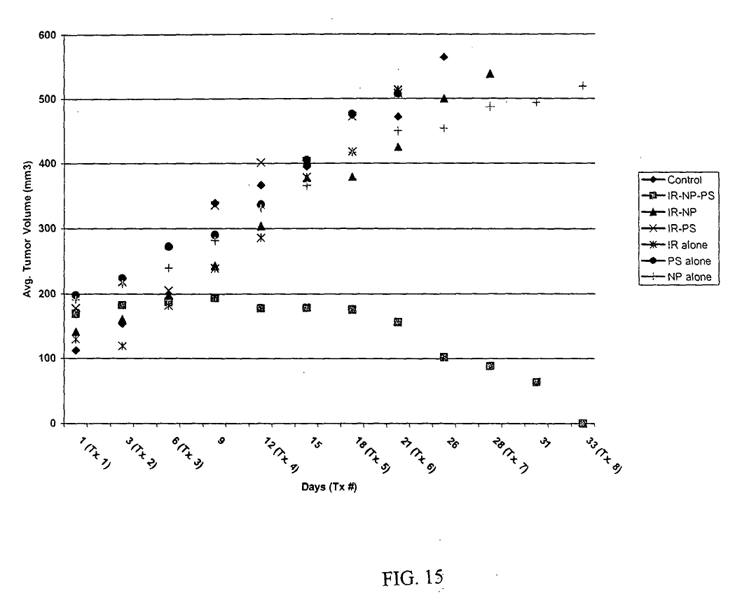

The IR-NP-PS group exhibited an initial decrease in tumor size after the third treatment, day 6, while the control groups experienced a steady rate of tumor growth with very little or no delay. All control groups receiving IR light exhibited a minimal delay in tumor growth with no significant difference between the groups. All controls and sham treatments exhibited less than 25% survival by day 26. The IR-NP-PS group exhibited significant tumor regression; by day 26, treatment #6, a small palpable tumor (<90 mm³) was present in 5/10 animals and 3/10 animals had no measurable or palpable tumor. By day 33, treatment #8, 8/10 animals receiving IR-NP-PS presented with no sign of disease. These results can be seen in FIG. 15. The two mice from the IR-NP-PS group that did not exhibit effective PDT-induced tumor regression eventually grew to the mandatory 500 mm³ endpoint and were euthanized. All remaining animals in each group were monitored for sixty days or until tumor volume endpoint of 500 mm³ was reached.

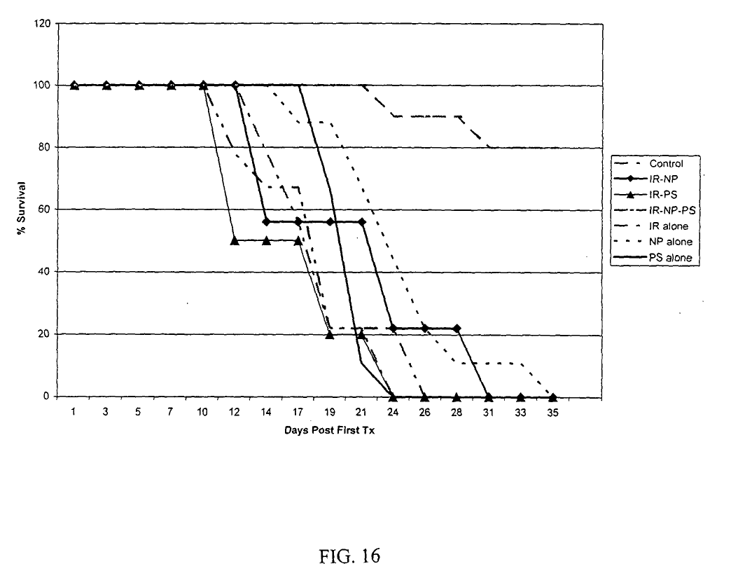

Upon completion of the sixty day observation period 80% of the IR-NP-PS treated animals survived, 8/10 of the animals exhibited complete tumor regression from an initial average tumor size of 169.5 mm³±61.70 (FIG. 16). The remaining two animals from the IR-NP-PS treated group exhibited very little PDT-mediated cytotoxicity. Tumors continued to grow to endpoint and by day 35 the animals were sacrificed. These two animals did however still exhibit a delayed tumor growth compared to control animals. No animals in the Control, IR-PS, IR-NP, IR alone, PS alone, and NP alone exhibited any tumor regression and all animals in the aforementioned groups grew to tumor endpoint. The total number of surviving animals from each group was as follows: Control—0/9; IR-NP-PS—8/10; IR-NP—0/9; IR-PS—0/10; IR alone—0/9; NP alone—0/9; and PS alone—0/9. Deaths related to anesthesia occurred in the IR-NP and IR alone treated groups, 1/10 mice for both groups. The Control, NP alone, and PS alone groups each had 1/10 mice that failed to develop a stable flank tumor prior to the first treatment and were subsequently removed from the study. This can be explained by human error during inoculations.

These proof-of-principle experiments are the first demonstration that “external beam” PDT is possible. By liberating photodynamic therapy from the constraint of needing to directly visualize and illuminate the target with visible light, the scope and applicability of PDT as a cancer treatment is substantially expanded. Our preliminary experiments have demonstrated that up-converting nanophosphors can efficiently excite photosensitizers and provide an effective method of PDT delivery stimulated by infrared light inducing tumor cell death and complete tumor regression.

### Example 10

Nanoparticles of upconversion phosphor, erbium- and ytterbium-codoped yttrium oxide, alkyl-coated with octodecyl phosphonic acid; coated with polystryene(1000)-block-polyethylene oxide(3000) as the stabilizer in THF and water made in a vortex mixer.

Upconversion phosphor nanoparticles, erbium- and ytterbium-codoped yttrium oxide (Y₂O₃:Er³⁺,Yb³⁺) are first coated with octodecyl phosphonic acid (ODPA), and thereby rendered hydrophobic and dispersible in organic solvents. Subsequently, the particles are mixed via Flash NanoPrecipitation with the block copolymer polystyrene(1000)-b-polyethylene oxide(3000), are coated with this copolymer during the mixing and thereby form particles that are colloidally stable in aqueous media.

0.020 grams of the above named surface-modified upconversion phosphor (UPC) nanoparticles, average diameter 160 nm, were resuspended in 4 mL tetrohydrofuran (THF) by probe-tip sonication for 15 min at 0° C. (Sonics and Materials Vibra-Cell, Power Setting 4). This suspension of UPC particles in organic solvent showed no visible signs of precipitation or aggregation even when the system was left unperturbed for several days. However, the same particles begin to precipitate instantly when mixed in DI water instead of THF.

An additional 6 mL THF were added to this suspension, and 0.1814 grams of an amphiphilic block copolymer, polystyrene(1000)-b-polyethylene oxide(3000), PS(1k)-b-PEO(3k), were dissolved in this mixture. The final composition of the organic solution consisted of 0.2 and 2.0 wt % of alkyl-coated upconversion phosphor and copolymer, respectively, in 10 mL THF. Part of this mixture was conserved for a “slow-mix” assay.

A four-stream vortex mixer (FIG. 1) was used to effect the coating of the UPC nanoparticles with the PS(1k)-b-PEO(5k). The organic solution was mixed at a 1:10 volume ratio with Milli-Q purified water with the described organic solution entering through one inlet streams, and Milli-Q water entering through the remaining three inlet streams. The flow rate of the PS(1k)-b-PEO(5k) and UPC particle containing organic stream was adjusted to 12 mL/min, while the three remaining streams of Milli-Q water were set a flow rate of 40 mL/min each, resulting in a total water flow rate of 120 mL/min. The solution was mixed and a sample collected for particle size and stability analysis.

The collected sample was clear to the eye and displayed no observable signs of aggregation or precipitation. Immediately after the mixed sample was collected, the size and size distribution of the particles were measured using dynamic light scattering (DLS). Particles showed an increase of 80-100 nm in average diameter, and the distribution curve of the coated particles was shifted uniformly to larger sizes compared to the distribution seen in DLS data of the particles before copolymer coating, indicating that the particles were successfully coated. Furthermore, the increase in particle size indicates that there was no significant aggregation of particles during mixing; the majority of the increase in diameter can be attributed to the polymeric coating.

When left undisturbed for a period of several days, the particles settled due, presumably, to the high density of the encapsulated metal; however brief and gentle agitation was sufficient for the particles to be completely and evenly redispersed. DLS measurements of the particles were taken at three times: immediately after mixing; one week after mixing; one month after mixing. It was observed that particle size and distribution remained effectively unchanged, indicating that the particles are colloidally stable in aqueous media for at least one month and display no aggregation during this time.

Two additional tests were performed to demonstrate that the obtained product of copolymer coated UPC nanoparticles that are colloidally stable in aqueous solution is process-specific. (1) Bare UPC nanoparticles, lacking the alkyl surface modification, were tested for their ability to be dispersed in organic solvent; this is a necessary preliminary step to do the mixing that allows the polymeric capsule to form. (2) Alkyl-coated UPC nanoparticles were mixed with block copolymer as per the above description with the single modification that mixing was done at a much slower rate in a test tube instead of at a high rate in a vortex mixer. Further details are given:

(1) This trial used pre-formed UPC nanoparticles from the same batch as the pre-formed UPC nanoparticles used above, with the sole distinction of not having undergone a process of hydrophobic surface modification by ODPA attachment. 0.0096 g of preformed UPC particles were mixed with 5 mL of THF. This corresponds to a composition of 0.21 wt %, which almost identically parallels the 0.22 wt % of alkyl-coated particles suspended in THF used for the successful copolymer mixing procedure detailed above. The mixture did not suspend well, and displayed both visible precipitate and visibly suspended particles.

UPC nanoparticles were probe-tip sonicated for 15 min. at 0° C. (Sonics and Materials Vibra-Cell, Power Setting 4) to achieve uniform re-dispersion, as was done with the ODPA-coated UPC nanoparticles. Immediately after sonication, particles appeared to be well dispersed; after 5 minutes a dark visible precipitate formed, and subsequent DLS measurement indicated that particle aggregation had occurred; the particles remaining in suspension displayed an average diameter of 1.2 microns. This indicates that the surface unmodified particles tended to aggregate even in organic solvent unlike the ODPA-coated UPC particles that displayed stability in an organic medium.

Further attempts to suspend these particles in DI water were also unsuccessful. 0.0010 mg of non-alkyl coated (“bare”) UPC nanoparticles were mixed in 5 mL of Pico-Pure water, i.e. a composition of 0.2 wt %. While no immediate precipitation was seen (unlike the ODPA-coated UPC nanoparticles that precipitated immediately upon suspension in water), DLS measurements showed the average particle diameter to be approximately 1.7 microns. After 15 hours, the particles had aggregated even further, showing visible precipitation.

As these trials show, the bare UPC nanoparticles are neither colloidally stable in organic media, as observed for the ODPA-coated UPC nanoparticles, nor are they colloidally stable in aqueous media, as seen with the ODPA-coated and PS(1k)-b-PEO(3k) encapsulated UPC nanoparticles. More importantly, due to their inability to be suspended in organic solvent, they cannot be coated via the Flash NanoPrecipitation method with amphiphilic block copolymers, and therefore cannot be stabilized in aqueous media. Thus, the process of attaching an alkyl coating to the surface is a crucial step towards aqueous stabilization of the UPC nanoparticles.

(2) This second assay demonstrates the necessity of the rapid mixing enabled by Flash NanoPrecipitation, as done with the vortex mixer. The organic mixture used in the polymer coating procedure above, consisting of 0.2 and 2.0 wt % ODPA-coated UPC nanoparticles and PS(1k)-b-PEO(3k), respectively, was also used for this “slow-mixing” test. 0.2 mL of this solution were mixed with 2 mL of Milli-Q water to preserve the 1:10 mixing proportion used in the rapid mixing with the vortex mixer. The organic mixture and Milli-Q water were micropipetted separately and simultaneously into a test tube. The test tube was then sealed with air trapped inside, and mixing was achieved by shaking the test tube vigorously for 15 s, followed by inverting the test tube 20 times. DLS measurement, taken once the mixture settled, indicated an average particle diameter of 630 nm.

This result of larger particle size indicates that the control of particle size afforded by the rapid mixing is lost when the mixing is done slowly; the particles aggregate either during or after mixing such that the initial particle size—which could be crucial to the intended application—becomes irrelevant. The long-term colloidal stability of these particles was not examined since particles of non-controlled size appear to be unusable.

### Example 11

Stable nanoparticles consisting of upconverting nanocrystals and a photosensitizer stabilized with block copolymer are synthesized through Flash NanoPrecipitation technology, using a multi-inlet vortex mixer as the mixing device. The upconverting nanocrystal in question is comprised of NaYF₄:Yb_{0.33},Er_{0.03} (with mean particle diameter of 230 nm as determined by Dynamic Light Scattering), the photosensitizer employed, meso-tetraphenyl prophine (mTPP), is selected from the group consisting of aminolevulinic acid, methyl ester of aminolevulinic acid and verteporfin. The amphiphilic block-copolymer used is methoxy(poly ethylene glycol-poly caprolactone) (mPEG-PCL). Briefly, the upconverting nanocrystals are initially suspended in tetrahydrofuran (THF) at 0.1 wt %. To this suspension, the photosensitizer and the block-copolymer are added such that their concentrations reach 0.1 wt % and 1.0 wt % respectively. This THF solution, which carries the actives, is then rapidly mixed against milliQ water in the MIVM. The organic stream (THF stream) bears a feed velocity of 12 ml/min while the water stream is fed at 108 ml/min. The resulting nanoparticles are immediately subjected to dialysis, which serves to arrest further growth of nanoparticles. Dialysed nanoparticles are stable for storage, with mean diameter of 300 nm.

The photodynamic therapy efficacy of these nanoparticles is tested in mice inoculated with AB12 malignant mesothelioma cells. Once the size of the tumor reaches ˜200-300 mm³, the nanoparticles in saline solution are administered via intra-tumoral injections. 10 mg of photosensitizer is administered per kg of mice weight. An infra red (IR) source at 980 nm with a maximum power of 1.0 W is utilized as an excitation source, with a total dosage of 28.3 J/cm². Pulsed light delivery method of 1 second ON, 2 seconds OFF is utilized to minimize tissue damage due to prolonged heating from the IR source. Regular treatment as described above results in tumor regression which is indicated by a significant decrease in tumor size and mice survival.

### Example 12

A set of nanoparticles bearing antibody on the particles' surface is tested for its targeted delivery behavior and efficacy. These nanoparticles consist of identical upconverting nanocrystals and photosensitizer as in Example 3. Functional block-copolymer (p-nitrophenylcarbonyl-PEG-b-PCL) are utilized in place of methoxy-(poly ethylene glycol-poly caprolactone). Similar nanoparticle formation procedure (MIVM followed by dialysis) is employed. The surface of the stable, functional nanoparticles is further reacted with anticancer monoclonal 2C5 antibody, which binds preferentially to tumor sites as well as localizes the upconverting nanocrystals and generated singlet oxygen at tumor sites. The monoclonal 2C5 antibody is attached to terminal p-nitrophenylcarbonyl (pNP) group of PEG-PCL stabilizing block-copolymers using the protocol of Roby, et. al (Eur. J. Pharm. Biopharm. 2006 April; 62(3): 235-240).

Mice inoculated with murine Lewis lung carcinoma B16 cancer cells are used in the test. Antibody-coated nanoparticles are administered intravenously and allowed to circulate. Similar IR dosage of 28.3 J/cm² are used as in example 1. Accumulation of antibody-coated nanoparticles is observed near the cancerous sites and tumor regression is observed upon periodic treatment.

### Example 13

Stable composite nanoparticles (CNPs) consisting of upconverting nanophosphors (UCNPs) and porphyrin, stabilized by biocompatible block-copolymers were synthesized using Multi Inlet Vortex Mixer (MIVM) via Flash NanoPrecipitation technology. The upconverting nanocrystal in question is comprised of NaYF₄:Yb_{0.33},Er_{0.03} (with mean particle diameter of 140 nm as determined by Dynamic Light Scattering), the photosensitizer employed is meso-tetraphenyl prophine (mTPP). Stabilizing block-copolymers candidates include (poly ethylene glycol-poly caprolactone) (PEG-b-PCL) and (poly ethylene glycol-block-poly lactic-co-glycolic acid) (PEG-b-PLGA). The UCNPs are initially suspended in tetrahydrofuran (THF) at 0.1 wt %. To this suspension, the photosensitizer and the block-copolymer (either PEG-PCL or PEG-PLGA) are added such that their concentrations reach 0.1 wt % and 6.0 wt % respectively. This THF solution, which carries the actives, is then rapidly mixed against milliQ water in the MIVM. The organic stream (THF stream) bears a feed velocity of 12 ml/min while the water stream is fed at 108 ml/min. The resulting nanoparticles are immediately subjected to dialysis, which serves to arrest further growth of nanoparticles. All of CNPs formulations (regardless of stabilizers) can be concentrated by ultrafiltration device equipped with a filter membrane and stir bar to keep the nanoparticles suspended or by centrifugation, without affecting its stability in water.

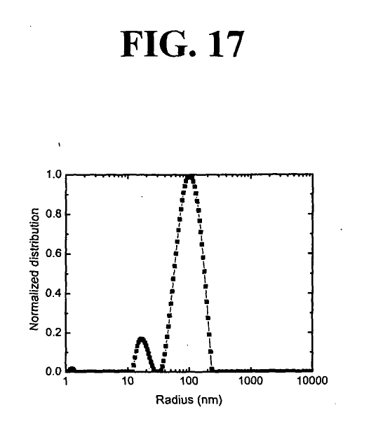

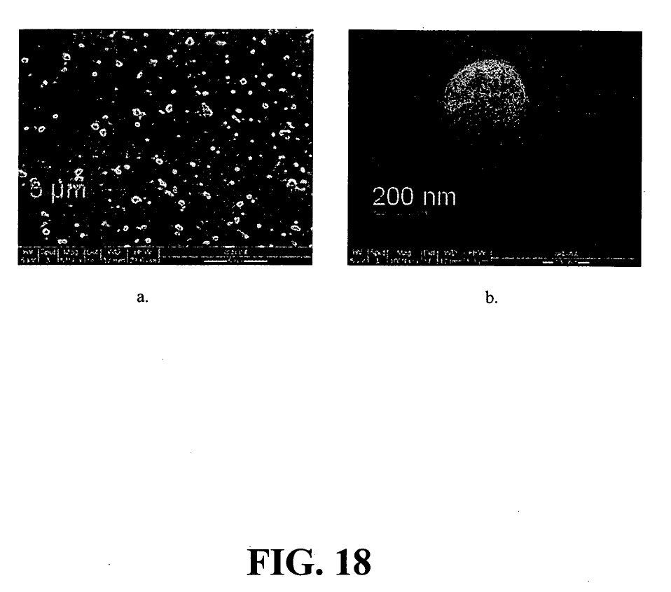

The synthesized particles were subjected to Dynamic Light Scattering for size analysis (FIG. 17) and to Scanning Electron Microscope (SEM) for size and morphology analyses (FIG. 18). PEG-PCL protected CNPs are stable in water with a mean radius of 100 nm (diameter=200 nm). The smaller peak present in the DLS size distribution represents a population of PEG-PCL protected mTPP micelles. SEM images confirm that the product is in the nanometer range, no micron-sized particles are observed. Composite nanoparticles (CNPs) are spherical in shape.

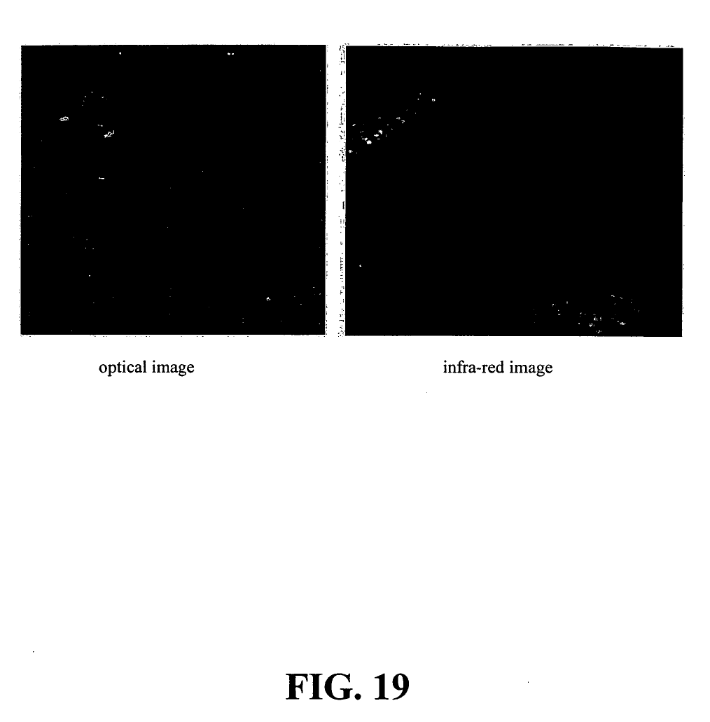

PEG-PCL protected CNPs were incubated with AB12 mesothelioma cells and exposed to IR. The images in FIG. 19 show that the PEGylated CNPs were uptaken by the cells. The image on the right is an optical image of mesothelioma cells incubated with nanometer-sized CNPs. The image on the left was captured when infra-red light was turned on, causing the UCNPs carried within the CNPs to light up in the cells.

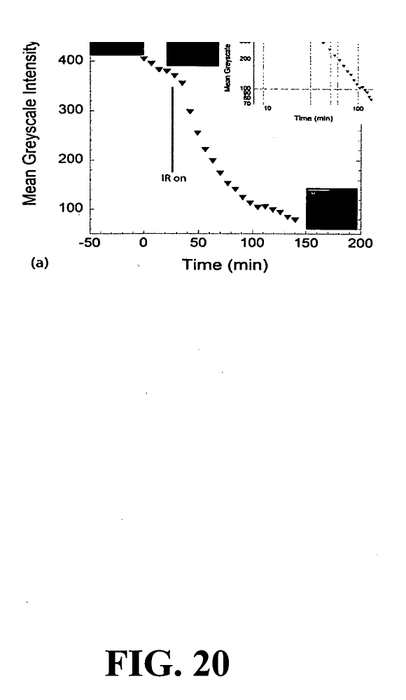

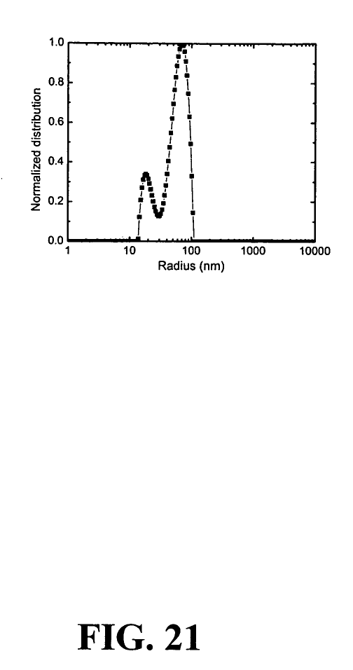

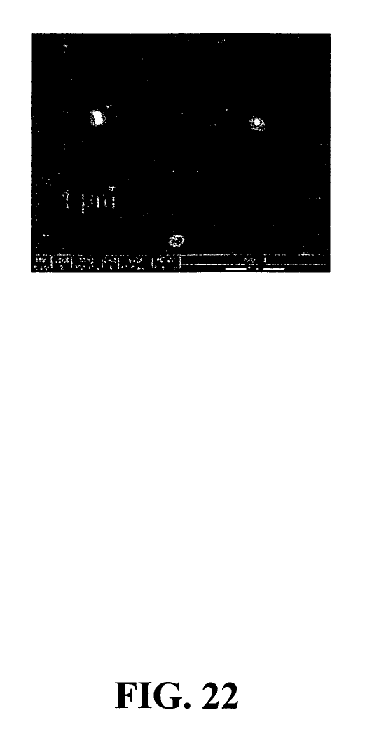

These CNPs are capable of producing cytotoxic singlet oxygen. When fluorescent anthracene-9,10-dipropionic acid (ADPA) reacts with singlet oxygen, non-fluorescent endoperoxide ADPA is formed. The production of singlet oxygen by IR (wavelength 980 nm, with a power of 20 Watt/mm²)-illuminated CNPs is investigated by monitoring the bleaching of ADPA. The results (FIG. 20) show that upon IR illumination, CNPs produce singlet oxygen, as seen in the subsequent reduction of ADPA fluorescence. Control experiment confirms that IR is necessary to activate the CNPs as no ADPA bleaching was observed when IR was turned off.

[symbol]

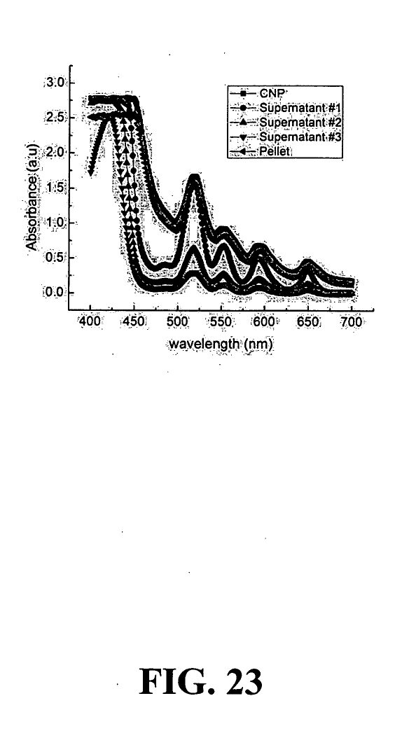

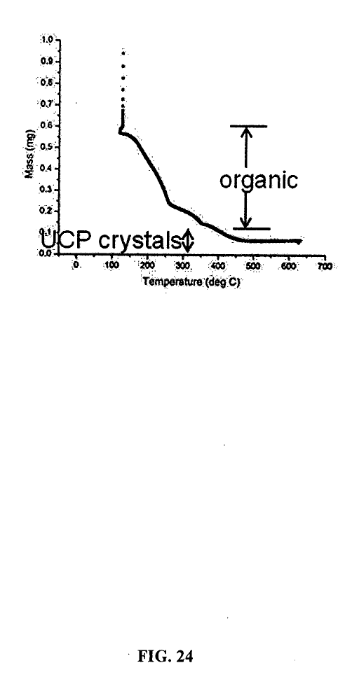

was performed 2 more times. The concentration of mTPP in every phase was determined by UV-Vis Absorbance (FIG. 23) and the concentration of UCNP crystals was determined (FIG. 24) by Thermogravimetric Analysis (TGA). The UV-Vis absorbance and TGA results show that the CNPs contain 1:3 ratio of mTPP:UCNP by weight. 25% of the feed TPP is incorporated within the CNPs while 75% forms PEG-protected micelles

Table of Upconversion Phosphors

TABLE 2

| | Lambda Exc. | |
|---|---|---|
| Host: Dopand(s) | (nm) | Lambda Em. (nm) |
| NaYF₄: Yb³⁺, Tm³⁺ | 975 | 450, 647, 698 |
| NaYF₄: Yb³⁺, Er³⁺ | 975 | 411, 540, 660 |
| NaYF₄: Yb³⁺, Ho³⁺ | 975 | 540, 648 |
| KYF: Yb³⁺, Tm³⁺ | 975 | 481, 652 |
| KYF: Yb³⁺, Er³⁺ | 975 | 550, 654, 670 |
| KYF: Yb³⁺, Ho³⁺ | 975 | 540, 648 |
| YLF: Yb³⁺, Tm³⁺ | 975 | 483, 648 |
| YLF: Yb³⁺, Er³⁺ | 975 | 541, 549, 654, 670 |
| YLF: Yb³⁺, Ho³⁺ | 975 | 544, 658 |
| LaF₃: Yb³⁺, Tm³⁺ | 980 | 800 |
| Y₂Si₂O₇ | 970 | 540-560, 670 |
| Y₂O₃: Yb³⁺, Er³⁺ | 980 | 416, 490, 535, 550, |
| | | 625, 665 |
| Y₂O₃: Er³⁺ | 980 | 416, 490, 535, 550, |
| | | 625, 665 |
| ZrO₂: Yb³⁺, Er³⁺ | 976 | 520, 540, 560, 655, |
| | | 675 |
| ZrO₂: Er³⁺ | 976 | 520, 540, 560, 655, |
| | | 675 |
| Na(Y_{1.5}Na_{0.5})F: Eu³⁺ | 980 | 470, 519, 653 |
| Er₃Al₅O₁₂ | 1480 | 380, 410, 456, 495, |
| | | 525, 550, 660 |

Table of Common Photosensitizers

TABLE 3

| | | Extinction |
|---|---|---|
| | Lambda Exc. | coefficient |
| Compound (or COMPOUND CLASS) | (nm) | 1/(M * cm) |
| 5-Aminolaevulinic acid as precursor to | 630 | |
| protoporphyrin IX (PpIX) | | |
| BACTERIOCHLORINS | >740 | |
| | (760 typical) | |
| Benzoporphyrin derivative mono-acid A | 650 | 34000 |
| (BPD) | | |
| Malachite green | 628 | 76000 |
| Merocyanine 540 | 560 | 138000 |
| Mg and Zn-tetrabenzoporphyrin | 630 | |
| Mono-L-aspartyl chlorin e6 | 654 | 40000 |
| (NPe6 or MACE) | | |
| m-tetra(hydroxyphenyl)chlorin | 652, 630 | 22400, 1172 |
| Nile Blue A | 620-650 | |
| PHENOTHIAZINIUM COMPOUNDS | | |
| ex: methylene blue | 668 | 74000 |
| ex: Toluidine blue | 630 | |
| PHORBIDES | 660-690 | |
| Photofrin | 630 | |
| PHTHALOCYANINES | ~680 | |
| PORPHYCENES | 635 | |
| PURPURINS | 630-715 | |
| ex: tin etiopurpurin (SnET2) | 700 | 40000 |
| Rhodamine 123 | 512 | 85200 |
| TEXAPHYRINS | | |
| ex: Cd-texaphyrin | 770 | |
| ex: Lanthanide(III) texaphyrins | 681-686 | |
| (Ln-Tex2+, Ln = La, Gd, Lu) | | |
| ex: Lutetium texaphyrin | 732 | |
| VERDINS | | |

1. A method comprising:
a) providing
(i) a composite nanoparticle comprising an upconversion phosphor and a photosensitizer,
(ii) a source of infra-red light, and
(iii) a cell
b) contacting said cell with said nanoparticle to create a contacted cell, and
c) exposing said contacted cell to infra-red light from said source.

2. The method of claim 1, wherein said nanoparticle further comprises a ligand and said ligand binds to said cell in step b).

3. The method of claim 1 wherein said nanoparticle enters said cell.

4. The method of claim 1 wherein, after step b), said composite nanoparticle releases an active oxygen species.

5. The method of claim 1 wherein said cell is in a live animal.

6. The method of claim 5 wherein said animal is a human.

7. The method of claim 5 wherein said cell is a cancer cell.

8. The method of claim 5 wherein said animal is suspected of having non-small cell lung cancer.

9. A system for killing cancer cells in vivo comprising:
a) an infrared-activable photosensitizer incorporated in a composite nanoparticle, said nanoparticle having an outer surface, said surface having attached thereto a ligand,
b) an upconversion phosphor incorporated in said nanoparticle,
c) a means for contacting said nanoparticle to a cancer cell, said cell having a binding site for said ligand, and
d) a means for exposing said cell to an infra-red light.
